(19) United States
(12) Patent Application Publication
(10) Pub. No.: US 2010/0216126 A1
(43) Pub. Date: Aug. 26, 2010

(54) MICROFLUIDIC DEVICE

(75) Inventors: Wamadeva Balachandran, Guildford (GB); Sayyed Mohamad Azimi, Uxbridge (GB); Jeremy Ahern, Ystradgynlais (GB); Massoud Zolgharni, Uxbridge (GB); Mohammad Reza Bahmanyar, West Drayton (GB); Predrag Slijepcevic, West Drayton (GB)

Correspondence Address: SENNIGER POWERS LLP, 100 NORTH BROADWAY, 17TH FLOOR, ST LOUIS, MO, 63102, US

(73) Assignee: BRUNEL UNIVERSITY, Uxbridge (GB)

(21) Appl. No.: 12/442,993

(22) PCT Filed: Jan. 12, 2008
(86) PCT No.: PCT/GB2008/000094

§ 371 Date: Apr. 29, 2010

(30) Foreign Application Priority Data
Jan. 12, 2007 (GB) 0700653.9

(51) Int. Cl. C12Q 1/68; B81B 7/02; G01N 33/50; G01N 1/28; G01N 1/34; C12M 1/00

(52) U.S. Cl. 435/6; 422/100; 422/82; 436/174; 435/289.1; 435/283.1

(57) ABSTRACT

A microfluidic device comprising; i) an inlet; ii) a first layer comprising at least first and second current carrying structures, wherein the at least first and second current carrying structures each comprise a plurality of teeth, and wherein the teeth of the first and second current carrying structures are optionally offset such that the teeth of the first current carrying structure are positioned between the teeth of the second current carrying structure; iii) a second layer comprising a first microfluidic chamber in fluid communication with the inlet positioned above the at least first and second current carrying structures of the first layer; and iv) a third layer comprising at least third and fourth current carrying structures wherein the at least third and fourth current carrying structures each comprise a plurality of teeth, and wherein the teeth of the third and fourth current carrying structures are optionally offset such that the teeth of the third current carrying structure are positioned between the teeth of the fourth current carrying structure; and wherein the at least third and fourth current carrying structures are positioned in the third layer so as to be above the first microfluidic chamber and such that the teeth of the third current carrying structure are positioned substantially vertically above or offset from the teeth of the first current carrying structure and the teeth of the fourth current carrying structure are positioned substantially vertically above or offset from the teeth of the second current carrying structure; wherein the teeth have a stem having substantially elliptical tip.

### SUMMARY

The current invention relates to a microfluidic device and to methods of its use for isolating and detecting an analyte from a biological sample.

### INTRODUCTION

Over the past decade, the advent of Micro-Electro-Mechanical Systems (MEMS) which is based on the miniaturization of mechanical components and their integration with micro-electrical systems, has created the potential to fabricate various structures and devices on the order of micrometers. This technology takes advantage of almost the same fabrication techniques, equipment and materials that were developed by semi-conductor industries. The range of MEMS applications is growing significantly and is mainly in the area of micro-sensors and micro-actuators. In recent years, miniaturization and integration of bio-chemical analysis systems to MEMS devices has been of great interest which has led to invention of Micro Total Analysis Systems (μ-TAS) or Lab-on-a-Chip (LOC) systems.

The main advantages of μ-TAS over traditional devices lie in lower fabrication costs, improvement of analytical performance regarding quality and operation time, small size, disposability, precise detection, minimal human interference and lower power consumption. Moreover, the problem of rare chemical and samples which restrain the application of genetic typing and other molecular analyses has been resolved by employment of μ-TAS.

However, whilst there has been a great deal of work in core areas, for example, miniaturizing PCR for expedited amplification of DNA in the microchip format, less effort has been exerted towards miniaturizing DNA purification methods. In fact, most of the currently demonstrated microfluidic or microarray devices pursue single functionality and use purified DNA or homogeneous sample as an input sample. On the other hand, practical applications in clinical and environmental analysis require processing of samples as complex and heterogeneous as whole blood or contaminated environmental fluids. Due to the complexity of the sample preparation, most available biochip systems still perform this initial step off-chip using traditional bench-top methods. As a result, rapid developments in back-end detection platforms have shifted the bottleneck, impeding further progress in rapid analysis devices, to front-end sample preparation where the “real” samples are used. A problem with the currently known microfluidic devices is performing efficient chaotic mixing in these platforms, this usually needs existence of moving parts, obstacles, grooves, and twisted or three dimensional serpentine channels. The structures of these components tend to be complex, however, requiring complicated fabrication processes such as multi-layer stacking or multi-step photolithography.

Suzuki, H., et al (J. microelectromechanical systems, 2004, vol 13, no.5 779-790) disclose a magnetic force driven chaotic mixer in which physical obstacles in the microchannel are used in conjunction with microconductors embedded in the base of the channel, which act to manipulate magnetic beads back and forth, to facilitate mixing of the sample and the beads.

EP 1462174 A1 discloses a device for controlled transport of magnetic beads between a position X and a position Y, wherein the beads are transported by applying successively a series of local magnetic fields generated by triangular current carrying structures in which the current density is non constant, resulting in the beads accumulating at the tips of the current carrying structures in the region having the highest charge density.

WO 2006004558 discloses a biochip for sorting and lysing biological samples which makes use of dielectrophoretic forces to retain and recover desired cells from a sample.

It is an object of the current invention to provide a microfluidic device which provides improved mixing of liquids, for example in a microchannel, or chamber and also provides simpler fabrication and which overcomes or mitigates the problems of the prior art particularly coagulation of whole blood samples.

### SUMMARY OF THE INVENTION

According to the present invention there is provided a microfluidic device comprising;

i) an inlet;
ii) a first layer comprising at least first and second current carrying structures, wherein the at least first and second current carrying structures each comprise a plurality of teeth, and wherein the teeth of the first and second current carrying structures are optionally offset such that the teeth of the first current carrying structure are positioned between the teeth of the second current carrying structure;
iii) a second layer comprising a first microfluidic chamber in fluid communication with the inlet and positioned above the at least first and second current carrying structures of the first layer; and
iv) a third layer comprising at least third and fourth current carrying structures wherein the at least third and fourth current carrying structures each comprise a plurality of teeth, and wherein the teeth of the third and fourth current carrying structures are optionally offset such that the teeth of the third current carrying structure are positioned between the teeth of the fourth current carrying structure;
and wherein the at least third and fourth current carrying structures are positioned in the third layer so as to be above the first microfluidic chamber and such that the teeth of the third current carrying structure are positioned substantially vertically above or offset from the teeth of the first current carrying structure and the teeth of the fourth current carrying structure are positioned substantially vertically above, or offset from the teeth of the second current carrying structure;
wherein the teeth have a stem having substantially elliptical tip.

In a variation of this aspect, is provided a microfluidic device comprising;

i) an inlet;
ii) a first layer comprising at least a first current carrying structure comprising a plurality of teeth;
iii) a second layer comprising a first microfluidic chamber in fluid communication with the inlet and positioned above the at least first and second current carrying structures of the first layer; and
iv) a third layer comprising at least a second current carrying structure comprising a plurality of teeth;
and wherein the second current carrying structure is positioned in the third layer so as to be above the first microfluidic chamber and such that the teeth of the second current carrying structure are positioned substantially vertically above or offset from the teeth of the first current carrying structure;
wherein the teeth have a stem having substantially elliptical tip.

It can be seen that this variation differs in so far as the first and third layers of the device each comprise a current carrying structure, rather than first and second, and third and fourth current carrying structures respectively. This however does not preclude the possible inclusion of further current carrying structures in the first and third layers.

The current carrying structure of either the first or the third layer may be orientated to include turns or changes in direction such that individual teeth of the structure may be orientated such that they are opposite one another. The individual teeth may also be offset from one another.

In the following discussion of the first aspect of the invention (which includes devices according to either variation as defined above) it will be understood that the preferred features described may be applied mutatis mutandis to either version of this aspect of the invention.

It will be understood that the term offset encompasses a range of possible spacings for the teeth of the first and second current carrying structures. The teeth may for example be spaced regularly and with the same spatial interval between teeth in the first and the second current carrying structure, although this need not be the case. The teeth of the first current carrying structure may for example be offset such that they are present halfway between the teeth of the second current carrying structure, or alternatively at another fraction of the distance between the teeth. The term offset also encompasses irregular spacing between the teeth of the current carrying structures and between the current carrying structures themselves.

Teeth will be understood to refer to projections along the path of the current carrying structure. The shape of each tooth may therefore comprise further shapes and structure, for example the stem portion of the projection may terminate in an elliptical tip.

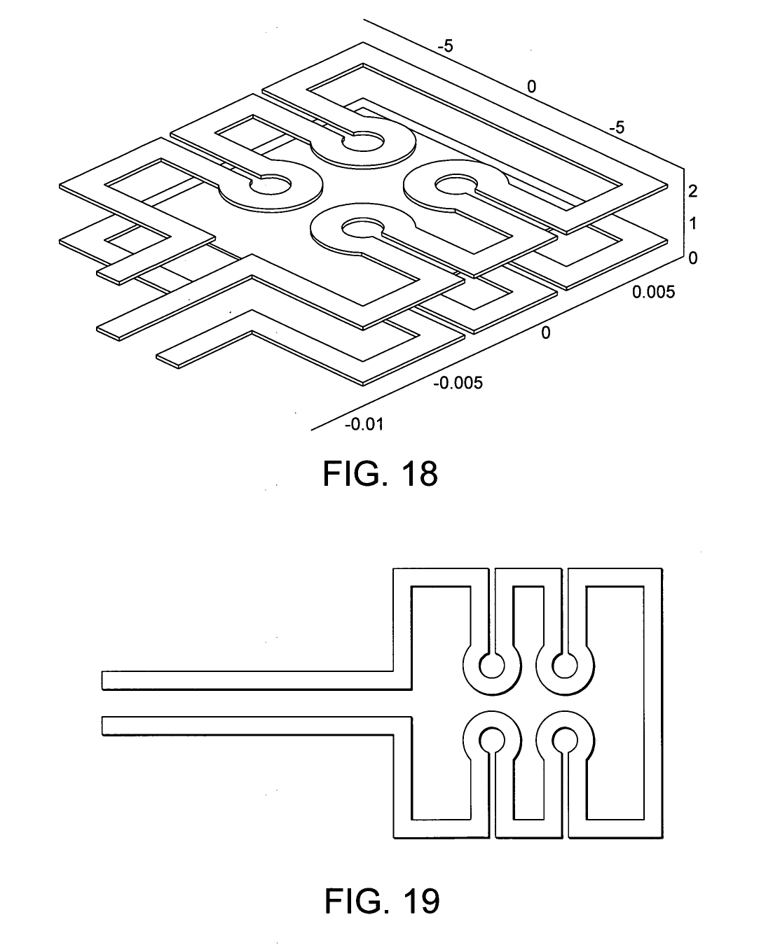

The current carrying structures may be of the kind described as “key-type” or “multiple turn key-type”. The spatial layouts of examples of such configurations are illustrated in FIGS. 18 to 20.

It will be understood that the term elliptical refers to a tip having an ovoid or circular conformation. In a preferred embodiment, the tip is circular.

The inventors have found that the elliptical configuration of the teeth of the device result in a magnetic field which is more evenly distributed about the tooth, as compared to other shapes of tooth, such as triangular, where the magnetic field is only stronger at the tip.

Preferably, the current carrying structures are embedded in the first and third layers. More preferably, the current carrying structures are between 0.1 μm to 10 μm below the surface of the first and third layers. Even more preferably, between 0.1 μm and 5 μm. Most preferably, between 0.1 μm and 2 μm.

It will be apparent to the skilled person that the device may also include a high permeable (e.g permalloy) layer located within or adjacent the first and/or third layers distal to the microchannel to increase the magnetic field generated by the device.

In a preferred embodiment, the first microfluidic chamber is a substantially straight channel. In a further preferred embodiment, the substantially straight channel has a region having increased dimensions forming a chamber proximal to the inlet.

It has been found that when device is in use, this region acts to increase the rate at which a sample liquid can be mixed. This is of particular use where the sample is a liquid which is liable to thicken or coagulate, for example whole blood. The use of blood as the sample is of particular interest in devices which are designed as home use or point of care use, because the sample can be easily obtained by a simple needle prick.

In a particularly preferred embodiment the inlet opens directly into the region having increased dimensions and the current carrying devices extend into this region such that chaotic mixing of the sample begins immediately the sample enters the device.

Preferably, the first and/or third layers further comprises a fifth current carrying structure. More preferably, the fifth current carrying structure is located so as to be distal to the inlet.

In a preferred embodiment the first microfluidic chamber forms a lysis and extraction unit. In one particularly preferred embodiment the device is useful for the analysis of whole blood.

Preferably, the microfluidic device further comprises a second microfluidic chamber in fluid communication with the first microfluidic chamber, wherein the second microfluidic chamber is an amplification chamber. More preferably, the amplification chamber is a PCR chamber.

It will be understood that the skilled person would be able to include the second chamber as such amplification chambers are well known in the art for example as described by Young, S. S., et al (J. Micromechanics and Microengineering, 2003 13; 768-774).

In a further embodiment, the microfluidic device comprises a third microfluidic chamber in fluid communication with the second microfluidic chamber, said third microfluidic chamber comprising a sensor for detecting the presence of an analyte.

In a particularly preferred embodiment, the sensor comprises a mutual inductance device.

In a yet further preferred embodiment, the microfluidic device comprises at least one integrated pump for effecting movement of a fluid from chamber to chamber. Preferably, the integrated pumps are magnetic pumps.

Preferably, the microfluidic device further comprises means for applying a voltage to each of the current carrying structures independently in a predetermined order and for a predetermined period.

Preferably, the period is in the range of 1-10 seconds, more preferably, less than 5 seconds.

Preferably, the microfluidic device further comprises at least a first fluid reservoir.

In one embodiment, the at least a first reservoir is in fluid communication with the first microfluidic chamber. Preferably, the at least first reservoir is integrated into the device.

In a further embodiment, the first microfluidic chamber forms the first fluid reservoir.

Preferably, the fluid comprises superparamagnetic beads.

More preferably, the fluid also comprises lysis buffer.

In a still further embodiment, the microfluidic device further comprising at least a second fluid reservoir.

It will be apparent that the fluid may comprise other constituents, for example, it may optionally comprise an anticoagulant.

According to a second aspect of the current invention, there is provided a lab-on-chip system for preparing a sample comprising a biological molecule, the system comprising;

a) the device according to the first aspect;
b) means for introducing the sample and the fluid into the first microfluidic chamber.

In a variation of this aspect, is provided a lab-on-chip system for preparing a sample comprising a biological molecule, the system comprising;

a) the device of the variation of the first aspect;
b) means for introducing the sample and the fluid into the first microfluidic chamber.

It can be seen that this variation differs in so far as the device concerned is the device according to the variation of the first aspect as described above.

In the following discussion of the second aspect of the invention (which includes systems that comprise either variation of the device of the invention) it will be understood that the preferred features described may be applied mutatis mutandis to either version of this aspect of the invention.

Preferably, the first, second, third and fourth current carrying structures of the device have a voltage applied thereto in a predetermined sequence.

In a preferred embodiment, a fifth current carrying structure acts to retain the superparamagnetic particles in the first microfluidic chamber.

It will be understood that the superparamagnetic particles may have any suitable diameter, preferably they have an average diameter from 50 nm to 10 μm. For example an average diameter of 3 μm is contemplated. Other diameters are possible.

Preferably, the superparamagnetic particles are functionalised so as to bind to an analyte of interest. More preferably, the analyte is a nucleic acid.

In a preferred embodiment the system further comprises a second reservoir containing a wash buffer in fluid communication with the first microfluidic chamber. Even more preferably, the system further comprises a third reservoir containing an elution buffer in fluid communication with the first microfluidic chamber.

It will be understood that the sample may be any suitable biological material. Preferably the sample comprises at least one cell. More preferably, the sample comprises a whole blood sample.

In a preferred embodiment, the fluid further comprises a lysis buffer.

In an even more preferred embodiment, the fluid further comprises an anticoagulant.

According to a third aspect of the current invention there is provided a method for the isolation of an analyte comprising a biological molecule from a sample, said method comprising the steps of:—

i) introducing the sample into the inlet of the device according to the first aspect:
ii) introducing a fluid comprising superparamagnetic particles into the first microfluidic chamber of the device;
iii) applying a voltage to the first, second, third and fourth current carrying structures of the device in a predetermined sequential order so as to cause electric currents to pass therethrough;
wherein, step i) can be performed prior to, concomitantly with or subsequently to step ii); and wherein, said superparamagnetic particles are functionalised so as to bind to the analyte of interest;
and wherein step iii) is performed concomitantly with or immediately after step i);
wherein said electric current causes the current carrying structures to become non-permanently magnetised resulting in magnetic actuation of said superparamagnetic particles in 3 dimensions within the microfluidic chamber, said magnetic actuation of said superparamagnetic particles resulting in chaotic mixing of said sample and said fluid resulting in an increased chance of the functionalised superparamagnetic particles coming in to contact with the analyte.

In a variation of this aspect, there is provided a method for the isolation of an analyte comprising a biological molecule from a sample, said method comprising the steps of:—

i) introducing the sample into the inlet of the device according to the variation of the first aspect:
ii) introducing a fluid comprising superparamagnetic particles into the first microfluidic chamber of the device;
iii) applying a voltage to the current carrying structures of the device in a predetermined sequential order so as to cause electric currents to pass therethrough;
wherein, step i) can be performed prior to, concomitantly with or subsequently to step ii); and wherein, said superparamagnetic particles are functionalised so as to bind to the analyte of interest;
and wherein step iii) is performed concomitantly with or immediately after step i);
wherein said electric current causes the current carrying structures to become non-permanently magnetised resulting in magnetic actuation of said superparamagnetic particles in 3 dimensions within the microfluidic chamber, said magnetic actuation of said superparamagnetic particles resulting in chaotic mixing of said sample and said fluid resulting in an increased chance of the functionalised superparamagnetic particles coming in to contact with the analyte.

It can be seen that this variation differs in so far as the device concerned is the device according to the variation of the first aspect as described above.

In the following discussion of the second aspect of the invention (which includes systems that comprise either variation of the device of the invention) it will be understood that the preferred features described may be applied mutatis mutandis to either version of this aspect of the invention.

As mentioned above, the elliptical configuration of the teeth of the device result in a magnetic field which is more evenly distributed about the tooth, as opposed to other shapes of tooth, such as triangular, where the magnetic field is stronger only at the tip. This results in greater mixing due to chaotic movement of the beads.

In a preferred embodiment the device further comprises a fifth current carrying structure, the fifth current carrying structure having a voltage applied thereto subsequently to step iii) wherein the superparamagnetic particles are attracted to and retained on the fifth current carrying structure through magnetic interactions.

Preferably, the current passing through each current carrying structure is in the range of 100 mA to 10 A. More preferably, 100 mA to 750 mA. Most preferably, less than 500 mA

In a preferred embodiment, the method comprises the further step of introducing a wash solution into the first microfluidic chamber of the device, preferably, once the superparamagnetic particles have been retained on the fifth current carrying structure.

The method optionally comprises the further step of introducing an elution solution into the first microfluidic chamber of the device.

In a preferred embodiment, the voltage is applied to each of the first, second, third and fourth current carrying devices for sufficiently long so as to allow the beads to move to a predetermined location in the first microfluidic chamber.

In one embodiment of the method of the third aspect the current carrying structures have the voltage applied in the order one, four, three, two. However, it will be apparent to the skilled person that the voltage can be supplied to the current carrying structures in any desired order so as to obtain optimum mixing of the fluid comprising the superparamagnetic particles and the sample.

In a preferred embodiment of the current invention the sample comprises at least one cell. More preferably, the sample is a blood sample.

Preferably, when the sample comprises at least one cell, the fluid further comprises lysis buffer and mixing of the sample with the buffer causes the cell to lyse.

Preferably, the analyte is a nucleic acid. More preferably, DNA.

The method of the third aspect preferably comprises the further step of detecting the presence of the analyte.

Preferably the velocity of flow of the sample through the first microfluidic chamber is in the range 20-100 μm/s.

According to a fourth aspect of the current invention there is provided a device for detecting the presence of an analyte in a sample, comprising;

i) a mutual inductor
ii) an insulating layer having a first surface adjacent the spiral mutual inductor and an opposed second surface,
ii) a sample contacting layer having a first surface having at least one probe immobilised thereon and a second surface opposed to the first surface and positioned so as to be adjacent the second surface of the insulating layer,
wherein the mutual inductor comprises a first coil and a second coil.

In a preferred embodiment the fourth aspect the mutual inductor comprises a circular coil spiral, a square shaped spiral coil, serpentine stacked-spiral coils, or a castellated stacked-type conductor.

In a preferred embodiment the first and second coils are positioned such that the first coil is positioned vertically above the second coil.

In another preferred embodiment, the first and second coils are interwound.

It will be understood by the skilled person that the presence of the analyte is detected by passing an alternating current through the first coil and monitoring the second coil for changes in induced voltage.

Preferably, the probe is a nucleic acid. More preferably, the probe is DNA.

In a preferred embodiment the device further comprises a suitable high permeability material layer, such as permalloy, located adjacent the spiral mutual inductor distal to the insulating layer.

Preferably, the insulating layer comprises silicon dioxide

It will be understood that the immobilisation layer may comprise any suitable material, for example, gold, agarose or Si₃N₄. Preferably, the immobilisation layer comprises gold.

According to a fifth aspect of the current invention, there is provided a method of detecting an analyte in a liquid sample, comprising the steps of;

a) bringing the sample containing the analyte into contact with magnetic beads functionalised so as to bind the analyte,
b) isolating the magnetic beads from the sample
c) bringing the beads into contact with the device of the fourth aspect, wherein the at least one probe immobilised on the sample contacting layer binds to the analyte so as to retain the magnetic beads at the surface;
d) measuring the variation in the inductance of the spiral mutual inductor,
wherein, an increase in the mutual inductance indicates the presence of the analyte in the sample.

Preferably, the analyte is a nucleic acid.

More preferably, the probe is a nucleic acid.

The magnetic beads may for example be paramagnetic beads.

The invention will now be described in greater detail with reference to the following figures, in which:—

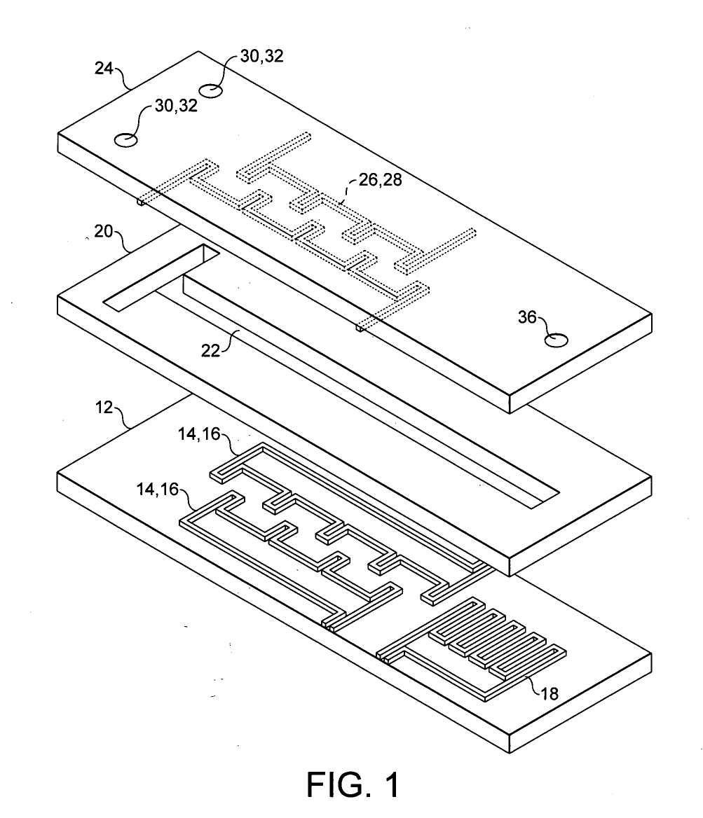

FIG. 1, is an exploded view of a microfluidic device according to the first aspect.

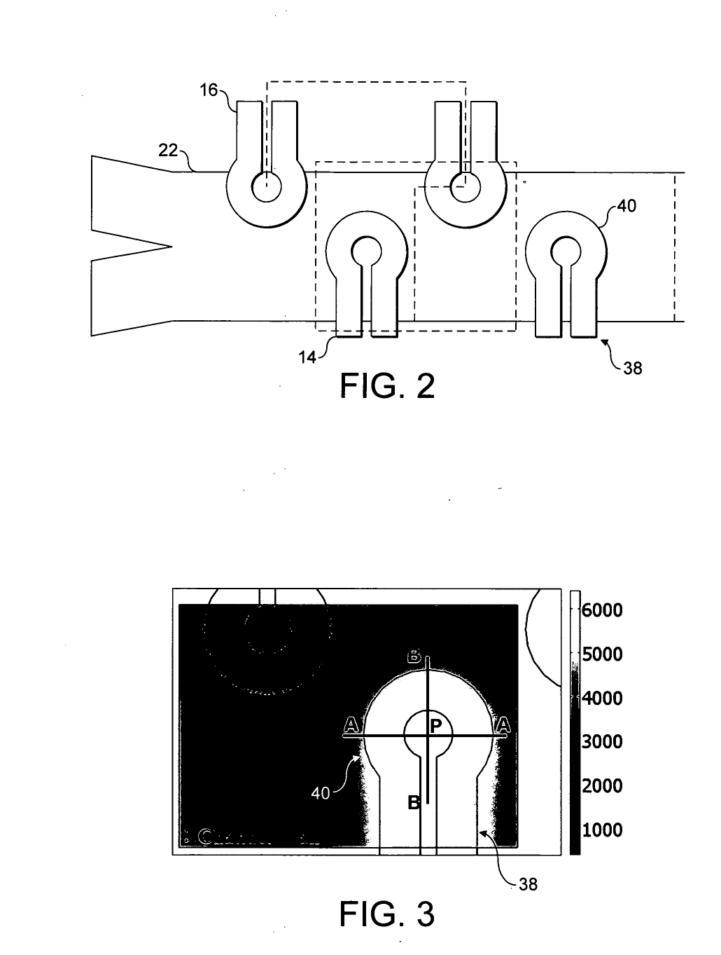

FIG. 2, shows a diagrammatic representation of the configuration of the current carrying structures forming one mixing unit in one layer of the device.

FIG. 3, shows one tooth of a current carrying structure showing the variation in magnetic field intensity.

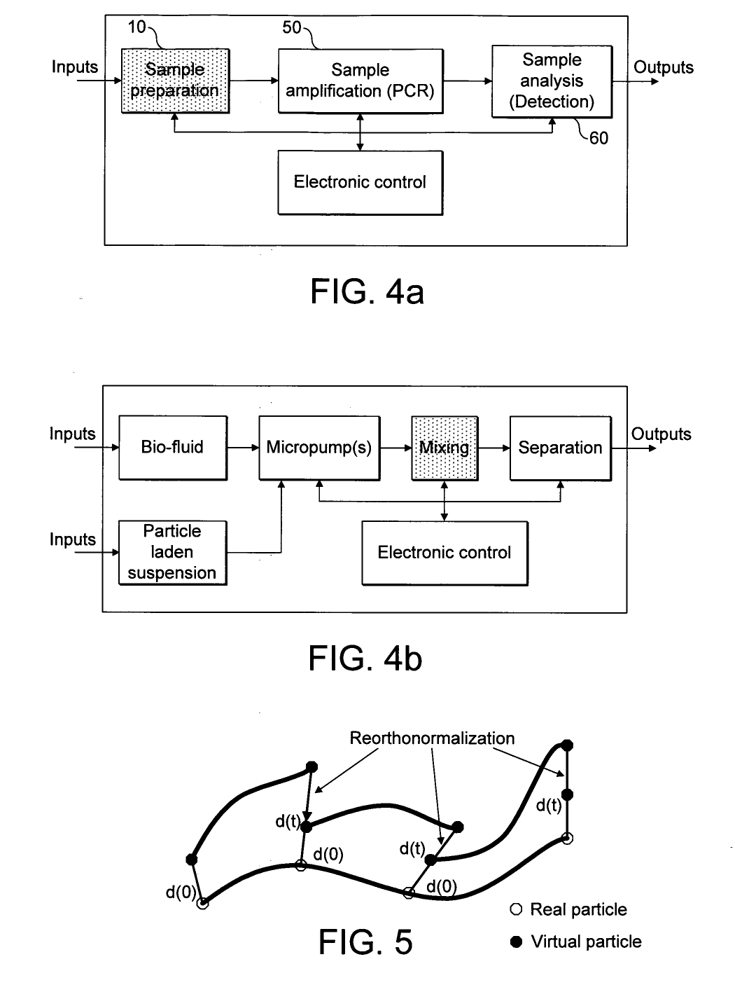

FIG. 4a, shows a diagrammatic representation of a lab-on-chip device comprising the microfluidic device according to the first aspect

FIG. 4b, shows a diagrammatic representation of an embodiment of the device according to the first aspect.

FIG. 5 shows a representation of Sprott's method for calculating the Lyapunov component.

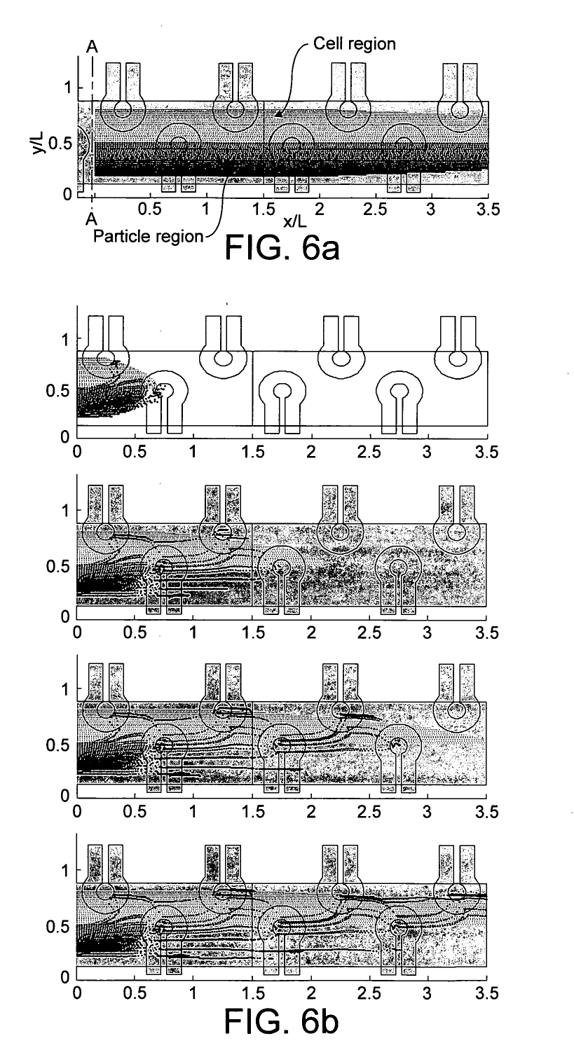

FIGS. 6a and 6b, show advection of cells within three and a half mixing units, a) without perturbation of cells and b) with magnetic perturbation.

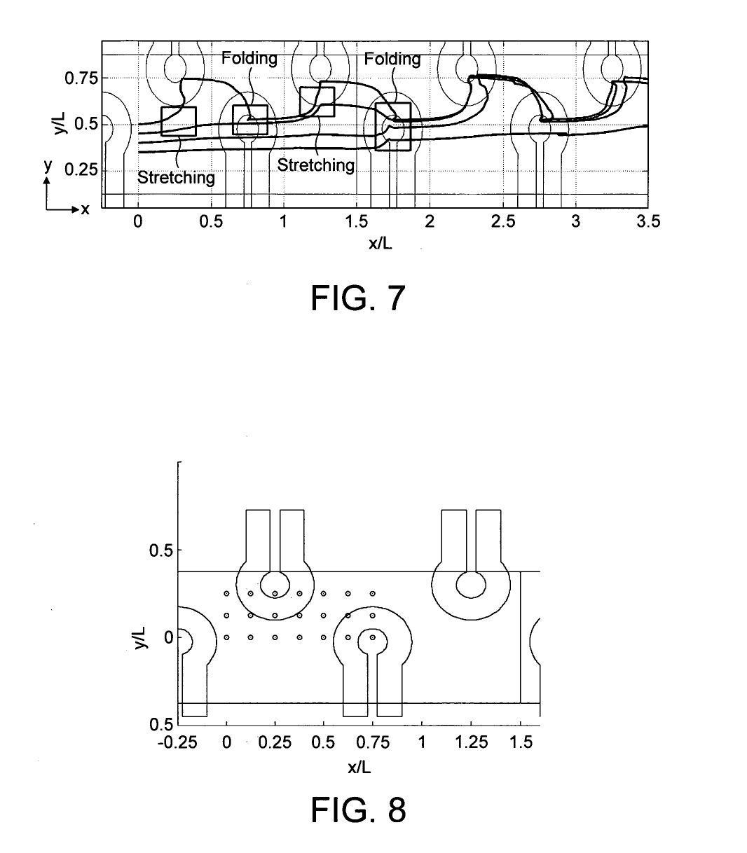

FIG. 7, shows simulated chaotic advection of four particles.

FIG. 8, shows the initial positions of individual particles for calculating the Lyapunov Exponent.

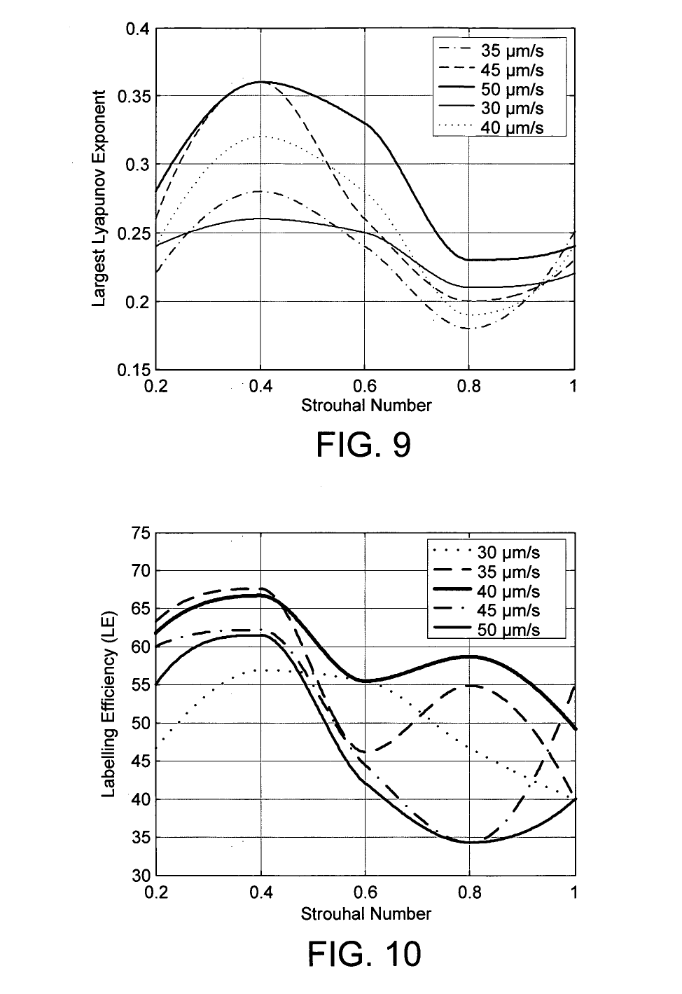

FIG. 9, shows the variation of largest LE against driving parameters

FIG. 10, shows the variation of labelling efficiency against driving parameters

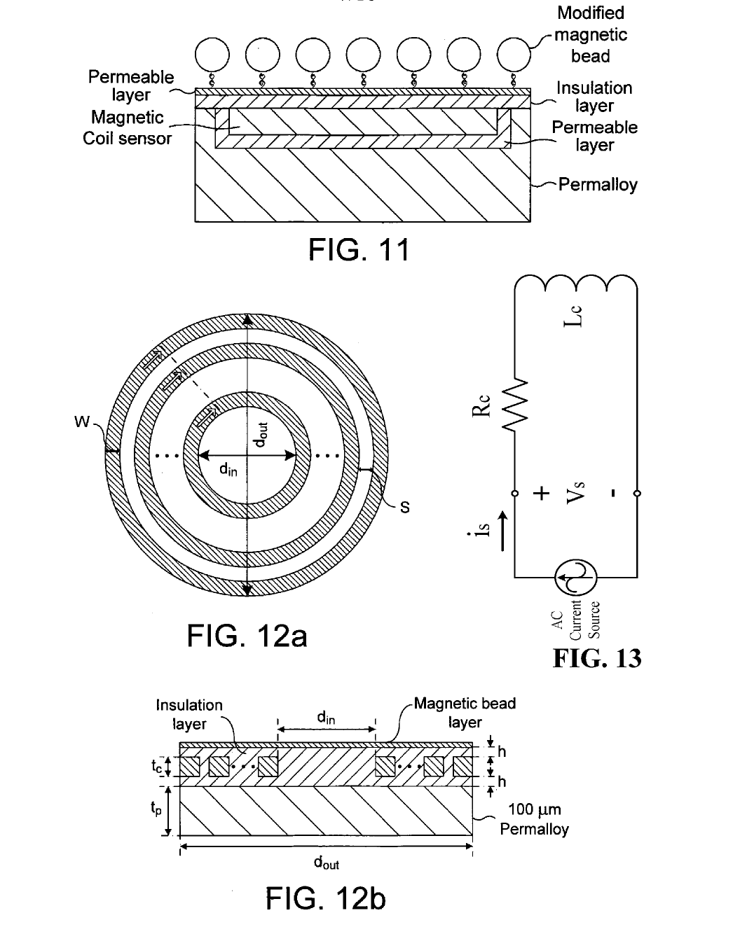

FIG. 11, shows a diagrammatic representation of the detector device according to the current invention showing hybridised DNA tagged with magnetic beads.

FIG. 12, shows a diagrammatic representation of the sensor model used in design simulations a) top view of coil, b) lateral cross section.

FIG. 13, shows an electrical model of the sensor.

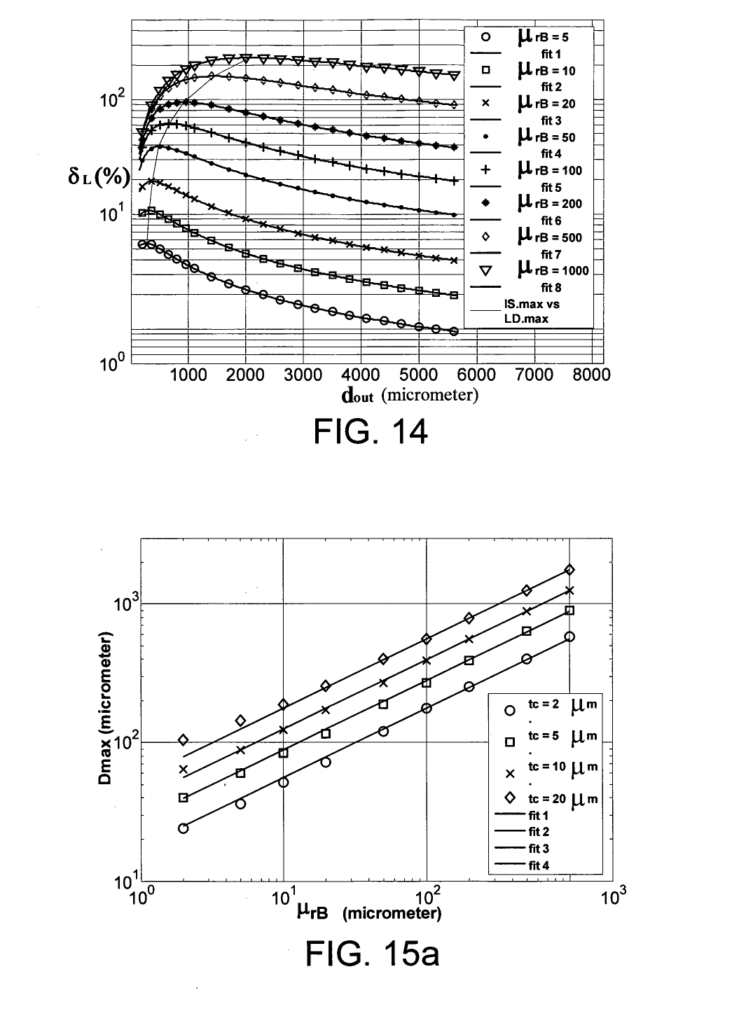

FIG. 14, shows the percentage change in coil inductance against outer coil diameter for different bead permeabilities.

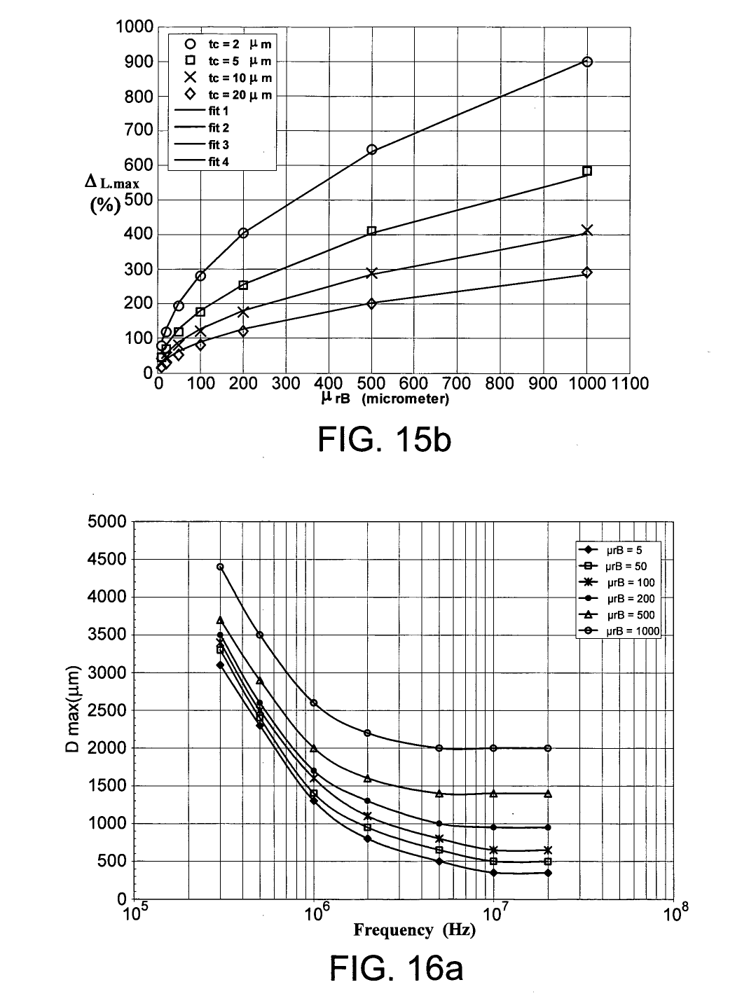

FIG. 15a, is a graph showing the optimal outer coil diameter at which output signal is maximised against bead permeability for different conductor thickness values.

FIG. 15b, is a graph showing the corresponding maximised inductance percentage change for the inductors of FIG. 15a.

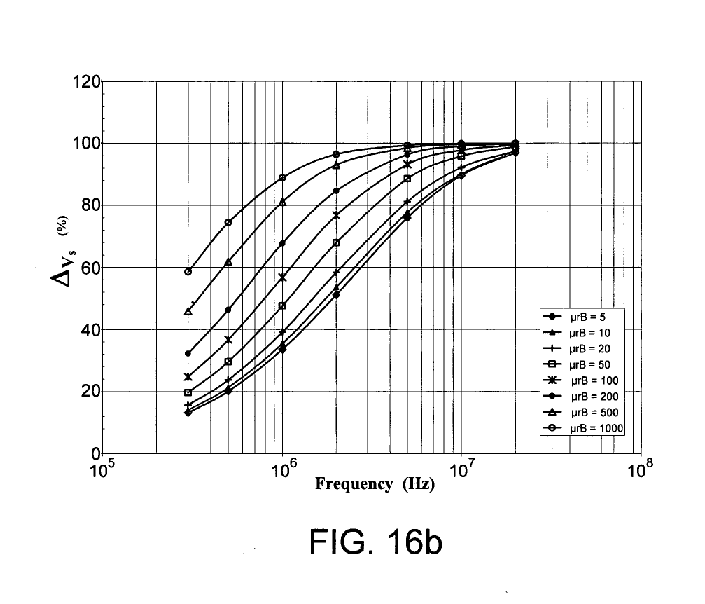

FIG. 16a, is a graph showing the optimal outer coil diameter at which output signal is maximised against bead permeability for different frequencies.

FIG. 16b is a graph showing the corresponding maximised sensor voltage for the frequencies of FIG. 16a

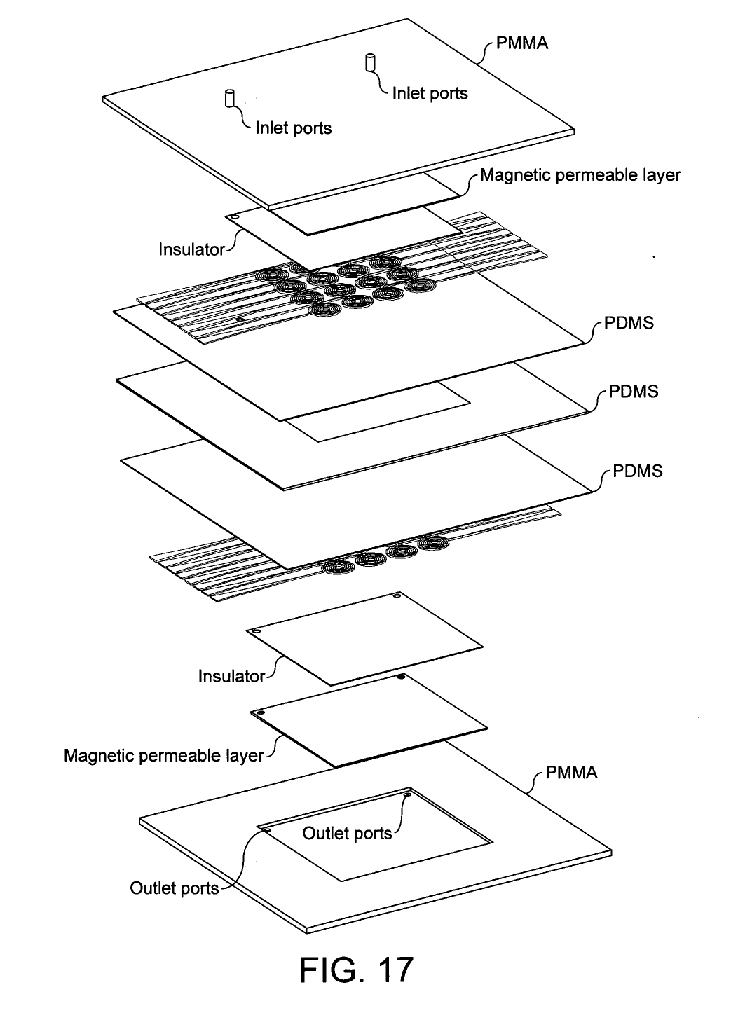

FIG. 17 shows a DNA extraction chip according to the present invention in exploded view.

FIG. 18 shows a 3 dimensional view of a key type electrode arrangement

FIG. 19 shows the dimensions of the key type electrode arrangement

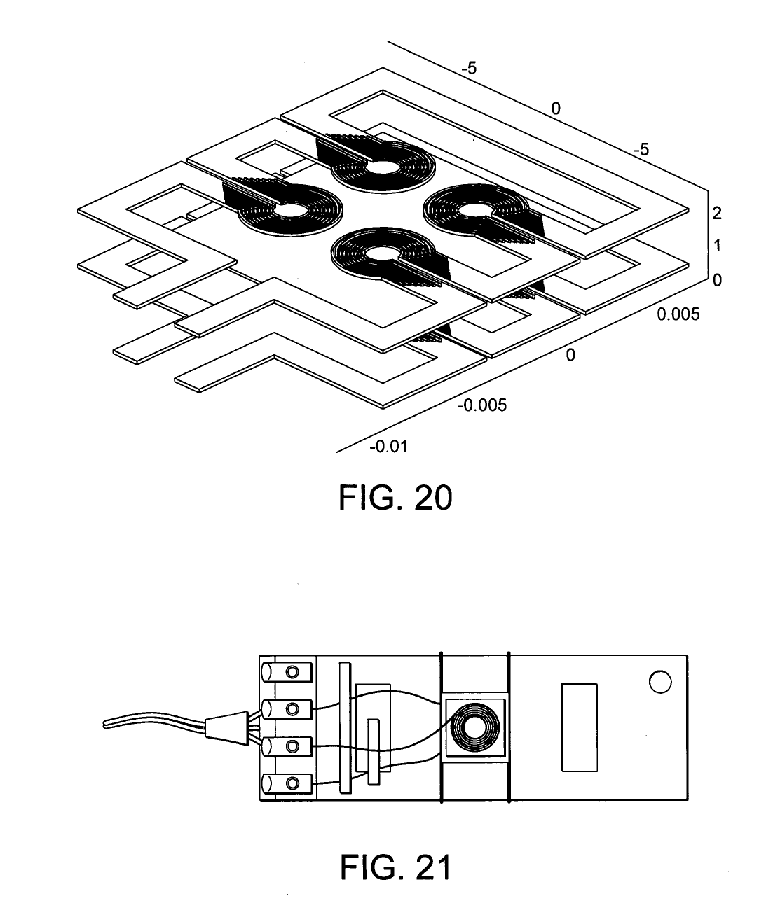

FIG. 20 shows a multiple-turn key-type electrode arrangement (dimensions: same as FIG. 19, except the width of each turn is 100 micrometers, inter-spacing between turns is 50 micrometers and thickness <100 micrometers)

FIG. 21 shows a photograph of the proof-of concept chip.

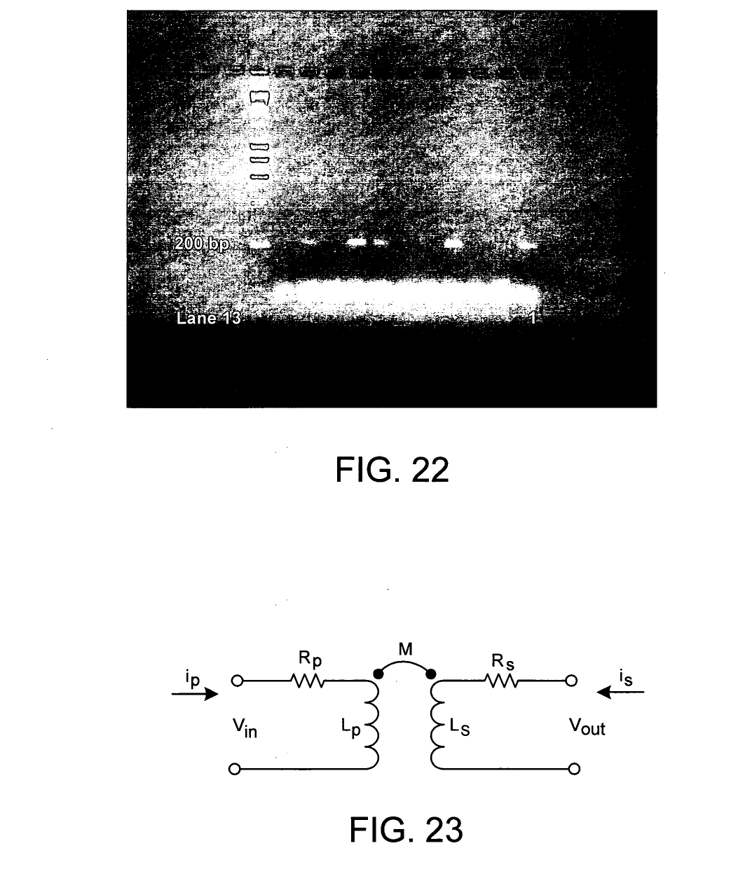

FIG. 22 shows the results of PCR performed on samples prepared using the proof of concept chip as shown in FIG. 21.

FIG. 23 shows an electrical model of a coupled inductor showing resistance and inductance of primary and secondary windings.

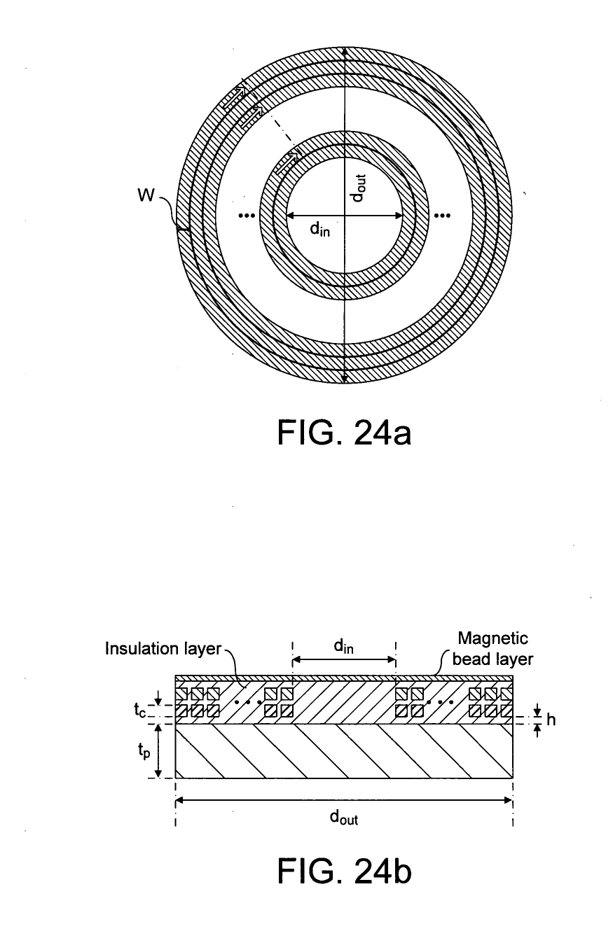

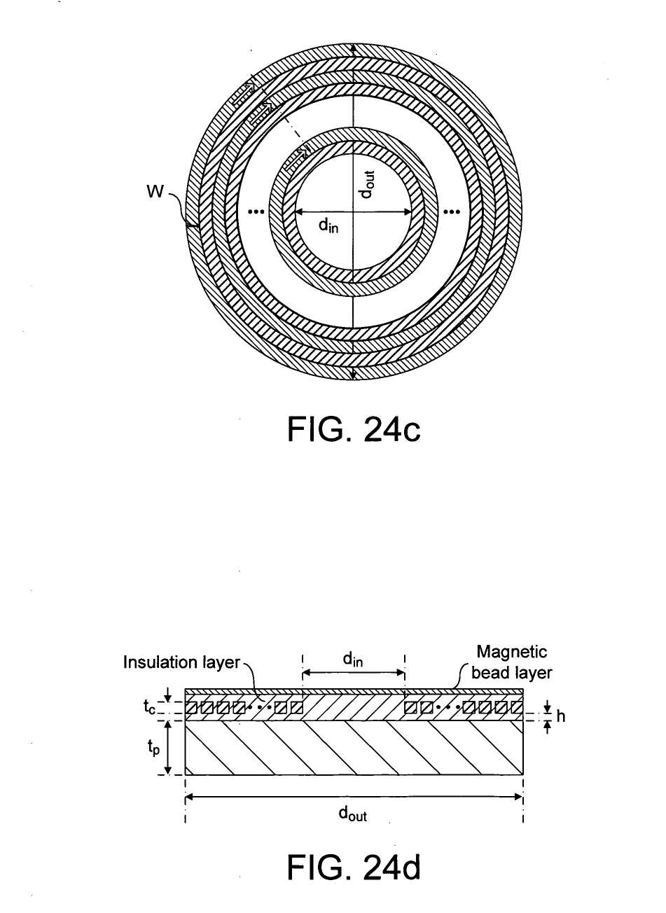

FIG. 24 shows Common types of planar coupled inductors [FIGS. 24(a)&(b) stacked-type windings, FIGS. 24(c)&(d) inter-wound windings]

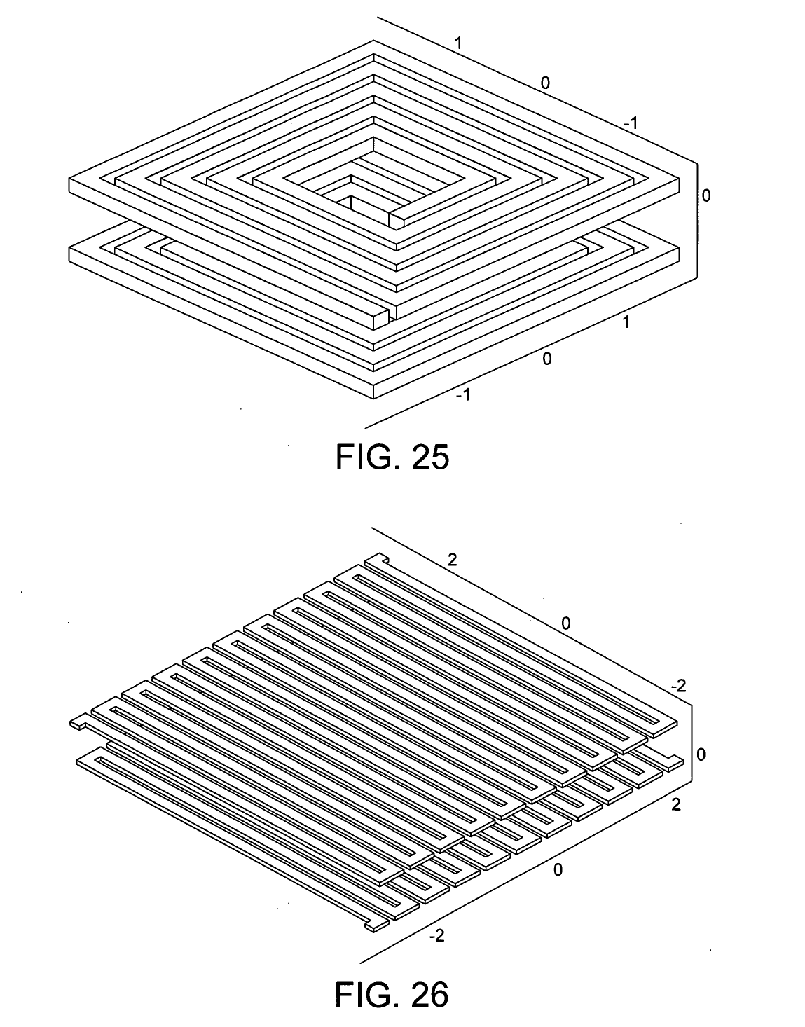

FIG. 25 shows square shaped stacked-spiral coils suitable for use as planar coupled inductors in the detecting device of the invention

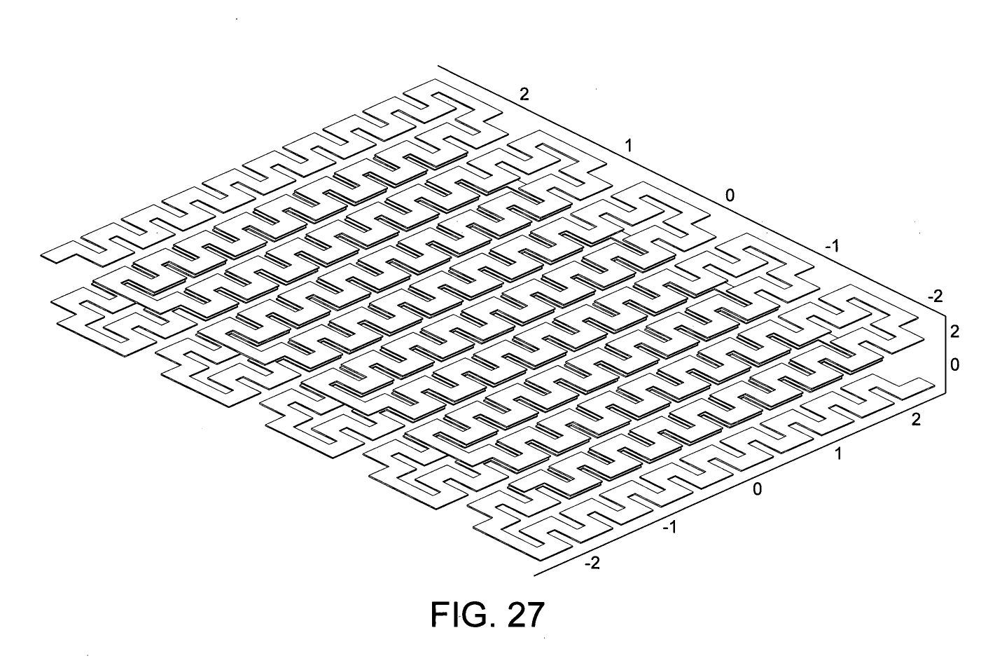

FIG. 26 shows serpentine stacked-spiral coils suitable for use as planar coupled inductors in the detecting device of the invention

FIG. 27 shows castellated stacked-type conductors suitable for use as planar coupled inductors in the detecting device of the invention

### DETAILED DESCRIPTION

The micromixer 10, as shown in FIG. 1 comprises a base layer 12 formed from glass having three serpentine conductors 14, 16, 18 embedded therein. A central layer 20 formed from PDMS comprising a straight channel 22 which is located above the serpentine conductors 14, 16, 18 and a upper layer 24 formed from glass having two further serpentine conductors 26, 28 embedded therein, two inlet ports 30, 32 and an outlet port 36.

An example of the dimensions of the device are shown in FIG. 2 where a top-view of one mixing unit with its boundaries is illustrated. Each mixing unit comprises two adjacent teeth from each conductor. Channel 22 is 150 μm wide and 50 μm deep. Conductors 14, 16 are in the shape of teeth 38 having circular tips 40 and are 35 μm high and 35 μm wide in the section and distances between centres of circular tips 40 of the conductors are 100 μm and 65 μm in x and y directions, respectively. Each row of upper and lower conductors 14, 16 is connected to the power supply alternately. The mixing operation cycle consists of two phases. In the first half-cycle, one of the conductor arrays in switched on while the other one is off. In the next half-cycle, the status of conductor arrays is reversed. Each mixing unit consists of two adjacent teeth 38 from opposite conductor arrays and the mixer is composed of a series of such mixing units which are connected together. In 3-D configuration, the switching between conductors will occur every 0.25 of a cycle.

FIG. 3 shows one tooth 38 with the magnetic field generated near the circular tip 40 of the conductor when a current of 750 mA is injected into one conductor array and is turned off in the opposite array during a half cycle of activation. The greyscale map represents variations in the magnetic field intensity at 10 μm above the surface of the conductor where the maximum magnitude of the field is about 6000 A/m at the centre of the circular tip (point P). The maximum force (5.5 pN) is applied on particles near the conductor and inside the circle of its tip where the intensity of magnetic field is at its maximum value. Although the magnetic field is maximum at the centre point P, the force on particles is relatively small at this point. This is due to the fact that the magnetic force is proportional to the gradient of the field which is almost constant in the neighbourhood of the point P. In moving away from the conductor, the force drops significantly due to a dramatic decrease in the magnetic field which in turn affects the magnetic moment.

The microfluidic device as shown in FIGS. 1 and 2 may be integrated into “lab-on-chip” devices such as those shown diagrammatically in FIGS. 4a and b. In FIG. 4a, the device comprises a sample preparation device 10, as shown in FIG. 1, linked in series to an amplification chamber 50 and a sample analysis unit 60 comprising a detector.

FIG. 4b, shows the sample preparation device 10 in greater detail. The device comprises an inlet to a micropump, linked to a mixing region and a separation region distal to the inlet.

Use of the lab-on-chip device as shown in FIG. 4 for the isolation and preparation of a DNA sample involves four steps including:
cell lysis
DNA binding
washing to purify/separate contaminants
resuspension

The first two steps are performed in the chaotic mixer followed by downstream processes in separator. Firstly, human blood and particle laden lysis buffer are introduced to the device, e.g. into the microchannel, through two inlet ports, for example by direct injection, under gravity, by negative pressure applied downstream, or using external pumps or integrated micropumps. Mixing of the particles is performed by applying local and time-dependent magnetic field generated by micro-conductors to produce chaotic advection in the motion of the particles through magnetophoretic forces. The embedded high aspect-ratio conductors allow a relatively large current to generate strong magnetic fields to move magnetic particles. Conductors on both top and bottom glass wafers are required to perform an efficient spatial mixing. Using a proper concentration of particles in lysis buffer, chaotic advection of the particles can be transferred to the fluids pattern, therefore, mixing the lysis buffer and blood. During the mixing and cell lysis, released DNA molecules are adsorbed onto the particles' surface.

After the mixing, the whole solution is then flowed downstream and the intact DNA/particles are separated from other contaminants by using another serpentine conductor fabricated at the bottom of the channel. In embodiments that employ a chamber, the bottom coil (or coils) can be utilized for this purpose. This conductor is activated by a constant DC current and due to the generated magnetic field; particles are gathered at the bottom surface of the channel while other contaminants are washed out with flow. Subsequently, washing buffer is introduced into the channel, which washes and removes remaining contaminants. Finally, conductors are switched off and resuspension buffer is pumped into the system and the purified DNA/particles are resuspended in it. The sample can now be used directly for PCR as the DNA is released upon heating the DNA/particle complex above 65° C. as required by a standard PCR protocol.

### Chaotic Microfluidic Mixer Design

Functionalized nano and microparticlees or beads offer a large specific surface for chemical binding and may be advantageously used as a “mobile substrate” for bioassays and in vivo applications (Gijs 2004). Due to the presence of magnetite (Fe₃O₄) or its oxidized form maghemite (γ-FE₂O₃), magnetic particles are magnetized in an external magnetic field. Such external field, generated by a permanent magnet or an electromagnet, may be used to manipulate these particles through magnetophoretic forces and therefore result in migration of particles in liquids.

By virtue of their small size; ranging from 100 μm down to 5 nm (Pankhurst et al. 2003), particles lose their magnetic properties when the external magnetic field is removed, exhibiting superparamagnetic characteristics. This additional advantage has been exploited for separation of desired biological entities, e.g., cell, DNA, RNA and protein, out of their native environment for subsequent analysis, where particles are used as a label for actuation. Prior to separation of the bio-cell/particle complex from contaminants, magnetic particles should be distributed throughout the bio-fluidic solution which contains target cells. This is done by a mixing process which helps to tag the target with particles. In the next stage, only those cells attached to magnetic particles will be isolated in the separation process, while the rest of the bio-fluidic mixture remains unaffected by the magnetic force. Separation of particles in microdevices based on magnetophoretic forces has been reported in the literature (Choi et al. 2001; Do et al. 2004; Ramadan et al. 2006).

Nevertheless, in micro-scale devices where the Reynolds number is often less than 1, mixing is not a trivial task due to the absence of turbulence. In such circumstances, mixing relies merely on molecular diffusion. Diffusion coefficient for a dilute solution of relatively large spheres in small, spherical molecules is estimated by Stokes-Einstein equation as follows (Mitchell 2004):

$$D = \frac{{κ}_{B} T}{3 π μ d} \qquad ( 1 )$$

where K_{B} is Boltzmann's constant, T is the absolute temperature, μ is the dynamic viscosity of the solvent, and d is the diameter of diffusing particle. The diffusion time constant t is proportional to the diffusion distance squared (τ=L²/D) which can be up to the order of 10⁵ seconds for particles with 1 μm diameter dispersed in water solution diffusing a distance of 100 μm. Obviously, such a diffusion time is not realistic and some improving mechanisms need to be employed to facilitate the mixing process.

In order to enhance the diffusion process, (multi-) lamination with different types of feed arrangements has been used in passive micromixers (Koch et al. 1999). The idea is to reduce the diffusion length scale using narrow mixing channels. Split-and-recombine (SAR) configurations (Haverkamp et al. 1999) can also enhance mixing through splitting and later joining the streams. Such configurations create consecutive multi-laminating patterns and increase the interfacial area. However, one disadvantage of using lamination for mixing of the particle laden fluids is the high probability of clogging in narrow channels. Another approach is to generate chaotic advection either by fabrication of special geometries and structures (e.g., obstacles (Wang et al. 2002), 3D channels (Liu et al. 2000), and grooves (Stroock et al. 2002)) or by applying external forces (e.g., dielectrophoretic (Deval et al. 2002), electroosmotic (Lin et al. 2004), pressure (Deshmukh et al. 2000) and thermal (Tsai et al. 2002) fields) in passive and active devices, respectively. Chaotic advection increases interfacial area and, consequently, enhances the mixing process. Recently, in addition to separation, magnetophoretic forces are exploited to enhance the mixing of the particles in solution (Rida et al. 2003); Rong et al. 2003; Suzuki et al. 2004). Here we describe the design of a chaotic magnetic particle-based micromixer and a numerical model in order to characterize the device with different driving parameters. To this end, a combination of electromagnetic, microfluidic and particle dynamics models has been used.

Conductors are utilized to produce magnetophoretic (hereafter, magnetic) forces and, therefore, chaotic pattern in the motion of particles and intensify the labelling of bio-cells. Two flows; target cells suspension and particle laden buffer, are introduced into the channel and manipulated by pressure-driven flow (see FIG. 2). While the cells follow the mainstream in upper half section of the channel (transported by convection of the suspending bio-fluid), the motion of magnetic particles is affected by both surrounding flow field and localized time-dependent magnetic field generated by periodical activation of two serpentine conductor arrays. Particles from various positions in the upstream and downstream sides are attracted towards the centre of the nearest activated tip where the maximum magnetic field exists. Chaotic patterns are produced in the motion of particles through utilizing a proper structural geometry and periodical current injection in conductors, thereby enhancing the spread of particles in the channel.

The magnetic force on particles is a function of the external magnetic field gradient and the magnetization of the particle. In de-ionized water, the magnetic force exerted on the particle in the linear area is described by:

$${r}_{p} = \int {V}_{p} \cdot d t = \int ( {V}_{f} + \frac{{F}_{m}}{3 π μ d} ) \cdot d t$$

where
d is the diameter of the spherical particle
H is the external magnetic field
Fₘ is magnetic force
μᵣ is relative permeability of the particle
μ₀ is permeability of the vacuum

Magnetic force is applied along the gradient of the external field and the particles are attracted towards higher magnetic field regions. Relative permeability and diameter of the reference particle used in this study (M-280, Dynabeads, Dynal, Oslo, Norway) are 2.83 μm and 1.76, respectively.

It should be noted that the magnetic force is three-dimensional and the z-component of the force is downward, which together with gravity, pull the particles towards the bottom of the channel and restrict their motion to a two-dimensional pattern. In fact, this component has no contribution to the chaotic motion of the particles and is assumed not to be influential on the process of mixing. Therefore, in this study planar forces close to the surface of the channel's bottom are of interest and simulation procedure is conducted on a two-dimensional basis.

Motion of the particles relative to the media can be assumed as a creeping flow and therefore, drag force on the particles can be evaluated by Stokes' law. The velocity of the particle due to the magnetic and drag forces can be described by Newton's second law:

$${m}_{p} \frac{\partial V}{\partial t} = {F}_{m} - 3 π μ dV , {V}_{m} = \frac{{F}_{m}}{3 π μ d} \qquad ( 2 )$$

where mₚ is the particle mass, V is the relative velocity of the particle with respect to the fluid, μ is the dynamic viscosity and d is the diameter of the particle. Term Vₘ is terminal velocity, which is reached by the particles after the exertion of the magnetic force. Particle relaxation time (τ=d²ρ/18μ) for the used particle (density of 1.4 g/cm³) and viscosity of water at room temperature (0.001 kg/ms), is in the order of 0.1 his. Therefore, acceleration phase in the motion is negligible and particle is assumed to react to the magnetic forces with no delay. Total velocity of the particle at each moment (Vₚ) would be the sum of the velocity due to fluid field (V_{f}) and the velocity due to magnetic field (Vₘ).

A two-dimensional numerical simulation is carried out assuming that there are no magnetic or hydrodynamic interactions between particles (one-way coupling) and motion of the particles is treated as if they are moving individually. This assumption is valid for small particles at low concentration in suspension, namely less than 10¹⁵ particles/m³ (C. Mikkelsen and H. Bruus, “Microfluidic capturing-dynamics of paramagnetic bead suspensions,” Lab Chip, vol. 5, pp. 1293-7, 2005) Newtonian fluid (water) field and time-dependent magnetic field are computed using commercial multiphysics finite element package Comsol (COMSOL, UK) and velocities of the particles due to these fields are extracted. Then trajectories of the particles are evaluated by integrating the sum of velocities using Euler integration method in Matlab:

$${r}_{p} = \int {V}_{p} \cdot d t = \int ( {V}_{f} + \frac{{F}_{m}}{3 π μ d} ) \cdot d t \qquad ( 3 )$$

Trajectories of the cells are obtained using the same Lagrangian tracking method with this exception that cells are magnetically inactive. Likely optimized structural geometry and dimensions (as mentioned earlier) and also a permissible current magnitude at which heat generation is not an issue (750 mA), obtained from preliminary studies are considered as constant parameters and two driving parameters; frequency of magnetic activation and flow velocity, are varied. Ratio of frequency to velocity is proportional to the dimensionless Strouhal Number (St):

$$St = \frac{fL}{V} \qquad ( 4 )$$

where f is the frequency, L is the characteristic length (here, distance between two adjacent teeth), and V is the mean velocity of the fluid. Simulations are conducted for the range of St=0.2-1. The size of biological entities may vary from a few nano-meters (proteins) to several micro-meters (cells). In this study, cells are considered to be spheres of 1 μm diameter. The bulk velocity of flow is in the order of 10 μm/s, which yields a Reynolds number of the order of 10⁻³, indicating that the flow is laminar.

In order to quantitatively evaluate the degree of mixing and efficiency of the system, two criteria are computed for the investigated range of simulation parameters. Efficiency of labelling of the target cells was used as the main index for characterizing the mixer. This method uses the idea of monitoring the trajectories of the particles and cells to predict their collision (if any) in the mixing domain (H. Suzuki, et al, “A chaotic mixer for magnetic bead-based micro cell sorter,” J. Microelectromech. Syst., vol. 13, pp. 779-90, 2004). It is assumed that collision happens when the distance between the centre of the spherical particle and cell becomes smaller than the sum of their radii, then cell is attached to the particle. Attachment of multiple cells to a single particle is possible and after each collision the trajectories of the particles must be recalculated using new free-body force diagram. Although the driving force is the same for the cell/particle complex (magnetic force is applied merely on the particles), the drag coefficients need to be modified according to the number of the attached cells. Subsequently, Labelling Efficiency (LE), i.e., ratio of the tagged cells to the total number of entered cells, is calculated over a specific period of mixing process.

Supplement to the stated index, largest Lyapunov exponent (λ) was used to quantify the chaotic advection of magnetic particles as a common definition of the mixing quality. Here Sprott's method (J. C. Sprott, Chaos and Time-Series Analysis, Oxford University Press, Oxford, 2003) is used to calculate the largest Lyapunov exponent (hereafter λ₁). This method utilizes the general idea of tracking two initially close particles, and calculates average logarithmic rate of separation of the particles. The numerical procedure is shown in FIG. 5. For any arbitrary particle, a virtual particle is considered with a minute distance of d(0) from the chosen particle and trajectories of these particles are tracked. At the end of each time-step, the new distance, d(t), between real and virtual particles and also the value of ln|d(t)/d(0)| are calculated. The virtual particle is then placed at distance d(0) along its connecting line to the real particle. After repeating this process for several time-steps, λ₁ will be converged and is evaluated by:

$${λ}_{l} = {lim}_{n \to ∞} \frac{1}{n Δ t} {\sum }_{i = 1}^{n} ln \frac{\vert {d}_{i} ( t ) \vert}{\vert d ( 0 ) \vert} \qquad ( 5 )$$

where Δt is the duration of one time-step and n is the number of steps. Examination of λ₁ for various particles reveals that generally after a period of 20 s, λ₁ approaches its converged value. Therefore, both indices of LE and λ₁ are calculated for a period of 20 s of mixing.

FIG. 6a illustrates the position of the particles and cells while advecting within three and half mixing units. Bio-cells (red dots, upper part of the diagram) and magnetic particles (blue dots, lower part of the diagram) enter the first mixing unit (across line A-A) from the left in upper and lower halves of the section, respectively, and with the same concentration. When there is no magnetic actuation, both cells and particles remain in their initial section and simply follow the streamlines of the parabolic velocity profile in Poiseuille flow. In this situation, tagging might occur only in the central region of the channel along the interface between two halves. All dimensions are normalized to the characteristic length.

FIG. 6b illustrates a typical effect of the magnetic actuation (St=0.4, V=40 μm/s) within the same mixing units at different snapshots. When the external field is applied, particles travel across the streamlines as well as the interface. Therefore, they find the opportunity to spread in upper section where they can meet and tag the cells. Magnetically inactive cells will have the same behaviour as previous situation when no perturbation was applied. As it can be observed, some particles far from the central line of the channel remain in the lower section as the magnetic forces in these regions are not strong enough to attract them during the lower array activated half-cycle.

In order to explain the basis for chaotic advection in the proposed micromixer, trajectories of four particles as shown in FIG. 7 are considered as typical trajectories in the mixer. Particles are released in the first mixing unit uniformly with the spacing of 10 μm when St=0.2 and V=45 μm/s. During the first half cycle, first array (conductor I) is on and second array (conductor II) is off. Particle I feels a strong magnetic force in y direction and tends to move in this direction while it is advected by the mainstream in x direction. Note that depending on its location in the channel which determines both drag force in the Poiseuille flow and magnetic force, particle I can have a positive or negative velocity in x direction. Particle 2 is farther from the conductor I and does not find any chance to be attracted upwards completely during the first half cycle. Therefore, two initially nearby particles diverge inducing the mechanism of stretching which is marked with a rectangle. In this phase particle I is exposed to the target cells across different streamlines and captures them in case on any collision.

In the following half cycle, electric current is injected into the conductor II and turned off in conductor I. In this phase, particle 1 is free to move from the previous location and is further advected by the mainstream until it approaches a region of strong enough magnetic force and, consequently, is pulled towards the centre of conductor II. Particle 2 is subject to a small magnitude of magnetic force in y direction but tends to move faster than the mainstream by virtue of magnetic force in x direction. In this phase, particle 2 approaches and tags the target cells, if any, along one streamline. Folding is achieved where two distant trajectories converge and even in some operating conditions cross each other. Consecutive stretching and folding can be produced in this way which is the basis of chaos.

Particles 3 and 4 which are too far from the conductor I to be attracted, are dragged downstream by the fluid and gradually move towards the upper half of the channel. After passing a few mixing units, almost all particles penetrate to cells' region and fluctuate in a chaotic regime confined to the tips of two conductors.

For computation of the largest Lyapunov exponent, 21 particles are uniformly distributed in upper half of the first mixing unit as the initial positions and (1 is calculated for each individual particle (see FIG. 8). The time period is 20 s when the particles approach their constant value of (1. In order to quantify the extent of chaos over the entire domain in the upper section (where cells exist), the average of (ls of 21 particles is taken. FIG. 9 illustrates variation of LE for different driving parameters (St=0.2-1) where each graph represents the values of LE for a constant fluid velocity (V=30-50 μm/s). Results for (l calculated over the same range of driving parameters are shown in FIG. 10. The global variations of (l is almost identical for different bulk flow velocities; the maximum chaos happens at St=0.4, while the minimum occurs at St=0.8. LE exhibits a similar behaviour at Strouhal numbers less than 0.6 which means that an increase in chaos leads to an increase in labelled cells.

Maximum values for (1 and LE are realized at St=0.4, which are 0.36 and 67%, respectively. At higher Strouhal numbers (namely 0.8), two indices show different variations. Although at high bulk flow velocities (larger than 40 μm/s) a good agreement between two indices can still be observed, in the case of lower velocities they show contradicting behaviours. At low flow velocities, some particles are advected until they are attracted to the centre of one tip in the upper conductor array. In the vicinity of the channel wall, flow velocity is much less than the central region of the channel. Since the magnetic forces are significantly large in the centre of the conductor, these particles will be stuck in this areas. Even after the current is switched to the opposite array, due to the low fluid velocity, particles will not have the opportunity to escape from the previous conductor. Therefore, in the next period, particles are dragged towards the same region hastily and again become trapped. In such operating conditions, the mixer is only partially chaotic, and the mixing is incomplete. However, trapped particles act like fixed posts, which may tag multiple cells, thereby increasing the value of LE. Although the efficiency is relatively high, in practice it is a challenging issue where trapped particles can clog the channel. However, when the Strouhal number is low, i.e., in case of longer time periods, particles have the chance to move away from these centres, even though the velocity is low.

A two-dimensional numerical study is performed in order to characterize the efficiency of the micromixer. Maximum labelling efficiency is found to be 67%. Lyapunov exponent as an index of the chaotic advection is found to be highly dependent on the Strouhal number where the maximum chaotic strength is realized in Strouhal numbers close to 0.4. It is shown that labelling efficiency in the mixer cannot be used as a stand alone index. Therefore, both indices need to be taken into account while characterizing the device.

### Fabrication of a Device According to the Present Invention

Devices according to the present invention (also known as chips) can be fabricated for example using basic building blocks in MEMS technology. MEMS technology has the ability to deposit thin films of materials on substrate, to apply a patterned mask on top of the films by photolithographic imaging, and to etch the films selectively to the mask. It is a structured sequence of these operations to form actual device.

The MEMS process starts with a rigid substrate material such as PMMA/Glass/Silicon/Polystyrene. On the top surface of the substrate, a high permeability layer (e.g. permalloy/Nickel) may for example be embossed or deposited using molecular beam process. An insulating layer of SiO2/PMMA/PDMS/Polystyrene may then be deposited on top of the permeable layer. The current carrying structure (also known as a coil structure) may be electroplated onto this surface using a mask and lithography. A thin layer of PDMS/PMMA/Polystyrene may then be spin coated on top of the coils to form a planar surface.

A microfluidic channel/chamber may for example be constructed using a pre-prepared PDMA/PMMA/Polystyrene cast of the desired thickness, for example of 150 microns and it is punched out of this sheet. This latter structure is sandwiched between two identical rigid substrate construction containing the coil electrodes and bonded using plasma bonding. The input and output ports may for example be punched or drilled through the structure.

### Proof of Concept Chip

The following discussion relates to embodiments of the invention as illustrated in FIGS. 17 to 22.

In these embodiments a central thin plane of an appropriately biocompatible material (e.g. PDMS), and of some 150 micrometres in thickness, has a central hole formed through it, preferably of rectangular shape. The length and width of this hole are calculated to give an appropriate final chamber volume, say 20 microlitres. This component formed the central part of the main lysis/mixing chamber and is closed by being sandwiched between two layers of similar or compatible material 10 to 100 micrometres in thickness. These cover-plates carry holes to allow inlet and outlet port-ways to the chamber thus formed.

Current carrying structures (i.e. a coil or coils) are placed on or in each of these thin layers, for example such that they are symmetrically disposed about the cavity. See FIGS. 17 to 20. Connections to these coils are brought out to the edges of this composite planar structure.

Such current carrying structures, when fed with appropriately switched currents, will cause a magnetic field to form and collapse normally to the principal plane of the cavity.

The magnetic field strength if further amplified by the introduction of a backing of an appropriately permeable magnetic material, such as a Permalloy alloy, nickel, mu-metal or similar. To prevent metallic contact between this layer and the planar electromagnetic coils, an insulating layer <100 micrometres in thickness is introduced.

Finally, the whole assembly is sandwiched between two outer plates of a material such as PMMA, which serves the following functions;

1) to give structural rigidity to the system
2) to act as an anchorage for inlet/outlet ports
3) to ensure a clean environment for the microfluidic and electrical network enclosed therein.

### Proof of Concept Results

The following discussion relates to the proof of concept chip as shown in FIG. 21 and the results obtained using said chip, as shown in FIG. 22.

4×10⁶ cell/ml in FCS and lysis buffer containing superparamagnetic beads (Dynabeads DNA Direct Universal Prod. No 630.06) were used to test this device. 10 microlitres of each was delivered directly into the lysis chamber via the inlet ports. Injected samples were subjected to one of the six following mixing conditions for 1 minute:

| | |
|---|---|
| no mixing (control) | |
| 50 mA | 4 HZ |
| 100 mA | 4 HZ |
| 150 mA | 4 HZ |
| 200 mA | 4 HZ |
| 200 mA | 0.2 HZ |

After each mixing condition, the DNA attached beads were collected from the lysis chamber, washed and the DNA adsorbed onto the beads was eluted by heating in a heating block for 5 minutes at 65° C. The supernatant (containing the eluted DNA) was removed using magnetic field. A PCR was performed on the samples. The hyperladder used was 1 Kb DNA extension ladder. The results obtained are shown in FIG. 22.

These results are summarised in the following table:

| Lane | setting | DNA amplified | Ng/band |
|---|---|---|---|
| 1 | No mixing | − | — |
| 3 | 50 mA 4 HZ | + | 20 |
| 5 | 100 mA 4 HZ | ++ | 28 |
| 8 | 150 mA 4 HZ | + | <15 |
| 9 | 200 mA 4 HZ | ++ | 28 |
| 11 | 200 mA 0.2 HZ | + | <15 |

Therefore, using the device according to the invention and the mixing conditions described above, lysis of cells occurred, subsequently enabling successful PCR. In contrast, under control conditions of no mixing, PCR was not successful. Thus the present inventors demonstrate successful microscale mixing and lysis of cells in less than one minute using a device according to the invention.

### Inductance Sensor Design

Traditionally, DNA hybridization detection is performed by using fluorescent tagging and optical read-out techniques. These techniques are efficient in conventional biology labs where specific protocols are followed by skilled technicians using expensive equipment. Moreover, conventional detection of DNA is a time consuming procedure which adds an extra cost to the whole process. To overcome these problems, considerable effort has been made for more than a decade to miniaturize and integrate the whole processes in a single disposable chip. Although detection of DNA by optical methods is reliable and well practised, it cannot be easily implemented on electronic chips. Alternative methods with potential for miniaturization have been investigated in recent years. Among these methods are electrochemical techniques (R. M. Umek et al., “Electronic detection of nucleic acids, a versatile platform for molecular diagnostics,” J. Molecular Diagnostics, vol. 3, pp. 74-84, 2001), piezoelectric sensors (T. Tatsuma, et al, “Multichannel quartz crystal microbalance,” Anal. Chem., vol. 71, no. 17, pp. 3632-3636, September 1999), impedance based techniques (F. Patolsky, et al, “Highly sensitive amplified electronic detection of DNA by biocatalyzed precipitation of an insoluble product onto electrodes,” Chemistry—A European Journal, vol. 9, pp. 1137-1145, 2003), and capacitance techniques (E. Souteyrand, et al, “Direct detection of the hybridization of synthetic homo-oligomer DNA sequences by field effect,” J. Phys. Chem. B, vol. 101, pp. 2980-2985, 1997). Micron-sized magnetic beads have also been widely used as labels in DNA detection (J. Fritz, et al, “Electronic detection of DNA by its intrinsic molecular charge,” Proc. Nat. Acad. Sci., vol. 99, no. 22, pp. 14 142-6, 2002) (L, Moreno-Hagelsieb, et al, “Sensitive DNA electrical detection based on interdigitated Al/Al2O3 microelectrodes,” Sens. Actuators B, Chem., vol. 98, pp. 269-274, 2004) (P. A. Besse, et al, “Detection of a single magnetic microbead using a miniaturized silicon Hall sensor,” Appl. Phys. Lett., vol. 80, pp. 4199-4201, 2002). Using magnetic beads allows easy manipulation of DNA and therefore may also facilitate mixing and separation protocols (D. R. Baselt, et al “A biosensor based on magnetoresistance technology,” Biosens. Bioeleectron., vol. 13, no. 7-8, pp. 731-739, October 1998) (J. C. Rife, et al “Design and performance of GMR sensors for the detection of magnetic microbeads in biosensors,” Sens. Actuators A, Phys., vol. 107, no. 3, pp. 209-218, 2003).

This example relates to a DNA hybridization detection sensor that uses magnetic beads attached to DNA strands as detectable particles. Increased concentration of magnetic beads due to DNA hybridization is detected in the form of inductance variations. The response of a planar spiral coil sensor to different types of magnetic beads is investigated and the effects of coil geometry as well as frequency on the performance of the sensor are numerically evaluated. Results and mathematical analysis provided for one coil can be extrapolated to multiple coils.

The sensor 100 of the current invention for DNA hybridization detection is illustrated in FIG. 11. The sensor 100 comprises a core 102 which is a planar spiral inductor which is sandwiched between an insulating layer 104 on the top and a layer of permalloy 106 in the bottom. The insulating layer 104 is covered with a permeable layer 108 to which probe DNAs 110 can attach and be immobilized. This layer could be any of standard surface treatments on gold coating or SiO₂—Si₃N₄. Magnetic beads functionalized with target DNAs 112 are applied to this surface. Specific Hybridization of target and probe DNA will result in formation of a layer of magnetic beads 112 above this surface 108. This layer is of high magnetic permeability and acts as one half of the magnetic core for the inductor. The underlying permalloy layer 106 acts as the other half of the magnetic core and completes the magnetic circuit. Formation of this magnetic circuit allows the magnetic flux to pass through easily and leads to an increase in the coil inductance. This property is used for detection of hybridization process.

### Parameters Affecting the Inductance

The inductance of the spiral coil is a function of various geometrical as well as physical parameters. The important geometrical parameters as depicted in FIG. 12 are defined as follows:

dₒᵤₜ: Coil outer diameter
dᵢₙ: Coil inner diameter
t_{c}: Conductor thickness
tₚ: Thickness of permalloy layer

The effect of interwinding distance S and the conductors thickness w are expressed in terms of fill factor (FF). The relative permeability of magnetic beads, μ_{rB} and the thickness of the bead layer t_{B}, formed after hybridization, are the physical parameters affecting the coil inductance.

### Electrical Model of the Sensor

The electrical model of the sensor is shown in FIG. 13. The coil is driven by an AC current source and the coil voltage is measured as the sensor output. After formation of the bead layer, the coil inductance is increased and the sensor output, Vₛ, will be changed. This amplitude of this voltage is used in order to detect the hybridization.

The amplitude of Vₛ can be expressed as follows:

Vₛ=\sqrt{R_{c}²+(ΩL_{c})²}Iₛ (1)

The voltage Vₛ is measured and its normalized variation is calculated to indicate the presence of the bead layer due to occurrence of hybridization. The frequency of the current source may be chosen in a range where R_{c} is constant. This means that for a particular sensor and source frequency, the voltage Vₛ is merely dependent on the inductance L_{c} and hence, the normalized variations of Vₛ may be calculated as follows:

$${δ}_{{V}_{s}} = \frac{{V}_{s} ( {L}_{c 2} ) - {V}_{s} ( {L}_{c 1} )}{{V}_{s} ( {L}_{c 1} )} \qquad ( 2 )$$

To understand the way δ_{V}_{ₛ} varies with respect to different geometrical and physical parameters explained above, the variations in L_{c} is computed numerically. This is then used to determine how the coil voltage will change in terms of different parameter values.

Based on the described concept, a three dimensional model of the sensor was simulated using the finite element package COMSOL FEMLAB Multiphysics v.3.2. Details of the model used in the simulation are shown in FIG. 12. The model was simulated for a layer of magnetic beads with effective thickness of 2 μm and different relative permeabilities. The normalized variations of the coil inductance, described in Equation 3, is computed numerically before and after hybridization and the results are presented in FIG. 14.

$${δ}_{L} = \frac{\begin{matrix}{L}_{c} ( {d}_{out} , {t}_{c} , {μ}_{rB} , {t}_{B} , {t}_{p} ) - \\ {L}_{c} ( {d}_{out} , {t}_{c} , {μ}_{rB} = 1 , {t}_{{ }_{B}} = 0 , {t}_{p} )\end{matrix}}{{L}_{c} ( {d}_{out} , {t}_{c} , {μ}_{rB} = 1 , {t}_{{ }_{B}} = 0 , {t}_{p} )} \qquad ( 3 )$$

The graphs of FIG. 14 show how δ_{L} changes with respect to the outer diameter dₒᵤₜ for different values of μ_{rB}. The values adopted for the other parameters are shown in Table 1.

Table 1: Various parameters and their corresponding values that are used in coupled inductors simulation.

| Parameter | Explanation | Quantity |
|---|---|---|
| t_{c} | Thickness of | 20 μm |
| | Conductor | |
| w_{c} | Width of | 20 μm |
| | Conductor | |
| s | Space Between | 30 μm |
| | Conductors | |
| FF | Fill Factor | %80 |
| | (occupied area of | |
| | conductors of the | |
| | coil to the total | |
| | coil area) | |
| h | Gap between coil | 10 μm |
| | and bead layer | |
| | which is occupied | |
| | with insulator | |

As shown in FIG. 14, for each value of the relative permeability, the sensor output is maximum at a specific value of dₒᵤₜ which may be denoted as Dₘₐₓ. It should be noted that the value of Dₘₐₓ is increasing with respect to μ_{rB} as shown by the dashed curve in FIG. 14.

To minimize the effect of permalloy on the signal, a very thick layer of permalloy (μᵣₚ=100 μm) has been used. Also a large space-domain (7 mm×14 mm) has been adopted in order to minimize computational errors.

In order to design a sensor with maximum response, it is useful to have the optimal coil diameter Dₘₐₓ in terms of different bead permeabilities and conductor thickness. The graphs in the FIG. 15a show the results of simulation for Dₘₐₓ in terms of μ_{rB} and t_{c}. Once the optimal diameter of the coil and the conductor thickness is known, it is useful to evaluate the magnitude of the output signal. These information may be derived from the graphs of FIG. 15b which depict the maximum change Δ_{L max}=δ_{L} (at Dₘₐₓ) corresponding to optimal values of Dₘₐₓ in terms of bead permeability and conductor thickness.

### The Effect of Frequency on Sensor Output

To see the behaviour of the sensor output with respect to frequency, the quantity δ_{V}_{ₛ} is computed for different bead permeabilities. The parameter values are as in Table 1 and the simulation results are shown in FIG. 16. For each value of the relative permeability and frequency, the sensor output is maximum at a specific value of dₒᵤₜ which is again denoted as Dₘₐₓ. The graphs of FIG. 16a show how these values are related to frequency. The corresponding sensor output Δ_{V}_{ₛ}=δ_{V}_{ₛ} (at Dₘₐₓ) which are normalized by

$${Δ}_{Lmax} = {lim}_{ω \to ∞} ( {Δ}_{{V}_{s}} )$$

are graphed in FIG. 16b.

### Variations on Sensor Design

A preferred embodiment used in the sensor utilizes a transformer arrangement. FIG. 23 shows a simplified model of a transformer. The series resistances of Rp and Rs are ohmic resistance of the conductors in the primary and secondary windings, respectively. Eqn. (1) shows the relationship between different parameters of the model.

$$( \begin{matrix}{V}_{out} \\ {V}_{in}\end{matrix} ) = ( \begin{matrix}{R}_{s} + {X}_{{L}_{s}} & - {X}_{M} \\ - {X}_{M} & {R}_{p} + {X}_{{L}_{p}}\end{matrix} ) ( \begin{matrix}{I}_{s} \\ {I}_{p}\end{matrix} ) \qquad ( 1 )$$

If the primary is connected to an AC current source and the secondary voltage is measured by a high impedance device, the secondary current Iₛ=0 and the Eqn. (1) reduces to:

Vₒᵤₜ=−X_{M}Iₚ (2)

Where X_{M}=ωM is the reactance due to the mutual inductance M. Eqn. (2) gives the secondary voltage which depends on X_{M} and I_{P}. Since I_{P} is constant, the measured secondary voltage is a direct measure of mutual inductance M. Mutual inductance may be expressed in terms of the self inductances of primary and secondary windings and coupling factor kₘ as follows:

M=kₘ\sqrt{LₚLₛ} (3)

If either of the transformer configuration of FIG. 24 is adopted, the primary and secondary self inductances are equal (Lₚ=Lₛ) and Eqn. (3) reduces to:

M=kₘLₚ (4)

Substituting M from Eqn. (4) into Eqn. (2) yields:

Vₒᵤₜ=−kₘX_{L}_{ₚ}Iₚ (5)

As is expressed in Eqn. (5), the output voltage is directly proportional to the primary (or secondary) reactance as well as the coupling factor km. Based on this result and through computer simulation, the output voltage is calculated for coils of different diameters and conductor thicknesses and optimum performance of the sensor has been obtained for magnetic beads of different permeabilities.

Simulations have been carried out for ideal full coverage of sensor surface with magnetic beads. If the magnetic bead coverage is partial then the output signal (ΔL.max) and Dmax will proportionately decrease.

In addition to the circular coil designs various other configurations of coil designs can also be utilized in the sensor. These are shown in FIGS. 25 to 27.

### REFERENCES

R. M. Umek et al., “Electronic detection of nucleic acids, a versatile platform for molecular diagnostics,” J. Molecular Diagnostics, vol. 3, pp. 74-84, 2001.
T. Tatsuma, Y. Watanabe, N. Oyama, K. Kitakizaki, and M. Haba, “Multichannel quartz crystal microbalance,” Anal. Chem., vol. 71, no. 17, pp. 3632-3636, September 1999.
F. Patolsky, A. Lichtenstein, I. Willner, “Highly sensitive amplified electronic detection of DNA by biocatalyzed precipitation of an insoluble product onto electrodes,” Chemistry—A European Journal, vol. 9, pp. 1137-1145, 2003.
E. Souteyrand, J. P. Cloarec, J. R. Martin, C. Wilson, I. Lawrence, S. Mikkelsen, and M. F. Lawrence, “Direct detection of the hybridization of synthetic homo-oligomer DNA sequences by field effect,” J. Phys. Chem. B, vol. 101, pp. 2980-2985, 1997.
J. Fritz, E. B. Cooper, S. Gaudet, P. K. Sorger, and S. R. Manalis, “Electronic detection of DNA by its intrinsic molecular charge,” Proc. Nat. Acad. Sci., vol. 99, no. 22, pp. 14 142-6, 2002.
L, Moreno-Hagelsieb, P. E. Lobert, R. Pampin, D. Bourgeois, J. Remacle, D. Flandre, “Sensitive DNA electrical detection based on interdigitated Al/Al2O3 microelectrodes,” Sens. Actuators B, Chem., vol. 98, pp. 269-274, 2004.
P. A. Besse, G. Boero, M. Demirre, V. Pott, and R. Popovic, “Detection of a single magnetic microbead using a miniaturized silicon Hall sensor,” Appl. Phys. Lett., vol. 80, pp. 4199-4201, 2002.
D. R. Baselt, G. U. Lee, M. Natesan, S. W. Metzger, P. E. Sheehan, and R. J. Colton, “A biosensor based on magnetoresistance technology,” Biosens. Bioeleectron., vol. 13, no. 7-8, pp. 731-739, October 1998.
J. C. Rife, M. M. Miller, P. E. Sheehan, C. R. Tamanaha, M. Tondra, and L. J. Whitman, “Design and performance of GMR sensors for the detection of magnetic microbeads in biosensors,” Sens. Actuators A, Phys., vol. 107, no. 3, pp. 209-218, 2003.
H. Suzuki, C. M. Ho, and N. Kasagi, “A chaotic mixer for magnetic bead-based micro cell sorter,” J. Microelectromech. Syst., vol. 13, pp. 779-790, 2004.
J. Do, J. W. Choi, and C. H. Ahn, “Low-cost magnetic interdigitated array on a plastic wafer,” IEEE Trans. Magnetics, vol. 40, pp. 3009-3011, 2004.
J. W. Choi, T. M. Liakopoulos, and C. H. Ahn, “An on-chip magnetic bead separator using spiral electromagnets with semi-encapsulated permalloy,” Biosens. Bioelectron., vol. 16, pp. 409-16, 2001.
Q. Ramadan, V. Samper, D. Poenar, and C. Yu, “Magnetic-based microfluidic platform for biomolecular separation,” Biomed Microdevices, vol. 8, pp. 151-8, 2006.
R. Rong, J. W. Choi, and C. H. Ahn, “A novel magnetic chaotic mixer for in-flow mixing of magnetic beads,” in Proc. Of the 7th Int. Conf. on Miniaturized Chemical and Biochemical Analysts Systems, California, 2003, pp. 335-8.
T. B. Jones, Electromechanics of Particles, Cambridge University Press, Cambridge, 1995.
C. Mikkelsen and H. Bruus, “Microfluidic capturing-dynamics of paramagnetic bead suspensions,” Lab Chip, vol. 5, pp. 1293-7, 2005.
J. C. Sprott, Chaos and Time-Series Analysis, Oxford University Press, Oxford, 2003.

1-63. (canceled)

64. A microfluidic device comprising;
i) an inlet;
ii) a first layer comprising at least first and second current carrying structures, wherein the at least first and second current carrying structures each comprise a plurality of teeth, and wherein the teeth of the first and second current carrying structures are optionally offset such that the teeth of the first current carrying structure are positioned between the teeth of the second current carrying structure;
iii) a second layer comprising a first microfluidic chamber in fluid communication with the inlet positioned above the at least first and second current carrying structures of the first layer; and
iv) a third layer comprising at least third and fourth current carrying structures wherein the at least third and fourth current carrying structures each comprise a plurality of teeth, and wherein the teeth of the third and fourth current carrying structures are optionally offset such that the teeth of the third current carrying structure are positioned between the teeth of the fourth current carrying structure;
and wherein the at least third and fourth current carrying structures are positioned in the third layer so as to be above the first microfluidic chamber and such that the teeth of the third current carrying structure are positioned substantially vertically above or offset from the teeth of the first current carrying structure and the teeth of the fourth current carrying structure are positioned substantially vertically above or offset from the teeth of the second current carrying structure;
wherein each tooth has a stem having a substantially elliptical tip.

65. The microfluidic device according to claim 64, wherein the current carrying structures are embedded in the first and third layers 0.1 μm to 10 μm below the surface of the first and third layers.

66. The microfluidic device according to claim 64, wherein the first microfluidic chamber is a substantially straight channel having a region of increased dimensions proximal to the inlet.

67. The microfluidic device according to claim 64, wherein the first and/or third layers further comprises a fifth current carrying structure located so as to be distal to the inlet.

68. The microfluidic device according to claim 64, wherein the first microfluidic chamber forms a lysis and extraction unit.

69. The microfluidic device according to claim 64, further comprising a second microfluidic chamber in fluid communication with the first microfluidic chamber, wherein the second microfluidic chamber is an amplification chamber which is a multiplexed PCR chamber.

70. The microfluidic device according to claim 64, further comprising a third microfluidic chamber in fluid communication with the second microfluidic chamber, said third microfluidic chamber comprising a sensor for detecting the presence of an analyte.

71. The microfluidic device according to claim 64, further comprising at least one integrated micropump, preferably a magnetic pump, for effecting movement of a fluid from one chamber to second chamber.

72. The microfluidic device according to claim 64, further comprising means for applying a voltage to each of the current carrying structures independently in a predetermined order and for a predetermined period.

73. The microfluidic device according to claim 64, further comprising at least a first fluid reservoir in fluid communication with the first microfluidic chamber and integrated into the device.

74. The microfluidic device according to claim 73, wherein the first microfluidic chamber forms the first fluid reservoir.

75. The microfluidic device according to claim 73, wherein the fluid comprises at least one of:
(a) superparamagnetic beads;
(b) lysis buffer; and
(c) an anticoagulant.

76. The microfluidic device according to claim 73, further comprising at least a second fluid reservoir.

77. A microfluidic device comprising;
i) an inlet;
ii) a first layer comprising at least a first current carrying structure comprising a plurality of teeth;
iii) a second layer comprising a first microfluidic chamber in fluid communication with the inlet and positioned above the at least first and second current carrying structures of the first layer; and
iv) a third layer comprising at least a second current carrying structure comprising a plurality of teeth;
and wherein the second current carrying structure is positioned in the third layer so as to be above the first microfluidic chamber and such that the teeth of the second current carrying structure are positioned substantially vertically above or offset from the teeth of the first current carrying structure;
wherein each tooth has a stem having a substantially elliptical tip.

78. A lab-on-chip system for preparing a sample comprising a biological molecule, the system comprising;
a) the device of claim 75;
b) means for introducing the sample and the fluid into the first microfluidic chamber.

79. The system according to claim 78, wherein in the first, second, third and fourth current carrying structures of the device have a voltage applied thereto in a predetermined sequence and a fifth current carrying structure acts to retain the superparamagnetic particles in the first microfluidic chamber.

80. The system according to claim 78, wherein the superparamagnetic particles have an average diameter from 50 nm to 10 μm and are functionalised so as to bind to an analyte of interest, which is preferably a nucleic acid.

81. The system according to claim 78, further comprising a second reservoir containing a wash buffer in fluid communication with the first microfluidic chamber.

82. The system according to claim 78, further comprising a third reservoir containing an elution buffer in fluid communication with the first microfluidic chamber.

83. The system according to claim 78, wherein the sample comprises at least one cell.

84. A method for the isolation of an analyte comprising a biological molecule from a sample, said method comprising the steps of:—
i) introducing the sample into the inlet of the device of claim 64;
ii) introducing a fluid comprising superparamagnetic particles into the first microfluidic chamber of the device;
iii) applying a voltage to the first, second, third and fourth current carrying structures of the device in a predetermined sequential order so as to cause electric currents to pass therethrough;
wherein, step i) can be performed prior to, concomitantly with or subsequently to step ii); and wherein, said superparamagnetic particles are functionalised so as to bind to the analyte of interest;
and wherein step iii) is performed concomitantly with or immediately after step i);
wherein said electric current causes the current carrying structures to become non-permanently magnetised resulting in magnetic actuation of said superparamagnetic particles in 3 dimensions within the microfluidic chamber, said magnetic actuation of said superparamagnetic particles resulting in chaotic mixing of said sample and said fluid resulting in an increased chance of the functionalised superparamagnetic particles coming in to contact with the analyte.

85. The method according to claim 84, wherein the device further comprises a fifth current carrying structure, the fifth current carrying structure having a voltage applied thereto subsequently to step iii) wherein the superparamagnetic particles are attracted to and retained on the fifth current carrying structure through magnetic interactions.

86. The method according to claim 84, wherein the current passing through each current carrying structure is in the range of 100 mA to 10 A, preferably less than 500 mA.

87. The method according to claim 84, comprising a further step of:
(a) introducing a wash solution into the first microfluidic chamber of the device;
(b) introducing a resuspension solution into the first microfluidic chamber of the device; or
(c) introducing an elution solution into the first microfluidic chamber of the device.

88. The method according to claim 84, wherein the voltage is applied to each of the first, second, third and fourth current carrying devices for sufficiently long so as to allow the beads to move to a predetermined location in the first microfluidic chamber.

89. The method according to claim 84, comprising the further step of detecting the presence of the analyte.
